(19) United States
(12) Patent Application Publication
(10) Pub. No.: US 2021/0329826 A1
(43) Pub. Date: Oct. 28, 2021

(54) PROCESSES FOR THE PREPARATION OF TREATED SEEDS

(71) Applicant: Monsanto Technology LLC, St. Louis, MO (US)

(72) Inventors: Darrell Allen Basler, Bloomsdale, MO (US); Damien Douglas Brewer, Creve Coeur, MO (US); Michael Francis Migliazzo, Chesterfield, MO (US); Daniel James Seyer, Ballwin, MO (US)

(73) Assignee: Monsanto Technology LLC, St. Louis, MO (US)

(21) Appl. No.: 16/479,816

(22) PCT Filed: Jan. 19, 2018
(86) PCT No.: PCT/US2018/014340

§ 371 Date: Jul. 22, 2019

Related U.S. Application Data
(60) Provisional application No. 62/585,112, filed on Nov. 13, 2017.
(60) Provisional application No. 62/508,601, filed on May 19, 2017.
(60) Provisional application No. 62/449,065, filed on Jan. 22, 2017.

(51) Int. Cl. A01C 1/06

(52) U.S. Cl. CPC A01C 1/06

(57) ABSTRACT

Provided herein are processes for the preparation of treated seeds. Generally, the processes described herein include providing a single, solid one-piece body. The single, solid one-piece body has a selected mass and volume. The process further includes reducing the single, solid one-piece body, and contacting the seeds with the seed treatment component.

### FIELD OF THE DISCLOSURE

The present disclosure generally relates to processes for the preparation of treated seeds.

### BACKGROUND

Prior to being planted, a seed may undergo seed treatment. For example, seeds may be treated with an active, such as amicrobial or fungicidal chemical, using a seed treater. Many seed treatment actives applied as coatings impart stickiness and/or tackiness to the seed surface. These surface characteristics can interfere with the flow of the seeds through industrial manufacturing and distribution equipment, which increases complexity and expense for the seed producer. For the same reason, seed treatment actives can also impede the flow of the seeds through planting equipment, which can result in planting errors and, ultimately, in lower crop yields.

In some cases, the flow of seeds can be improved by mixing the seeds with a powder flow-aid component, such as powdered graphite or talc, after treating with the active and before planting. However, due to heterogeneous volume density or powder packing fraction, the powder flow-aid component tends to respond in a variable fashion to processing equipment, most importantly in the treater feeding operation. Thus, the powder may lead to metering inconsistencies in the seed treater, including under-dosing, which may lead to ineffective lubricating of the seeds, and over-dosing, which may lead to undesirable dust-off and waste.

The seeds may be treated with other types of seed treatment components, other than or in addition to actives and flow-aid components.

### SUMMARY OF THE DISCLOSURE

In one aspect, a method of preparing treated seeds generally comprises providing a single, solid one-piece body. The single, one-piece component has a selected mass and volume. The method further comprises reducing the single, solid one-piece body, and contacting the seeds with the reduced single, solid one-piece body.

A treated seed is provided, wherein the seed is produced using a method as described herein.

Other features will be in part apparent and in part pointed out hereinafter.

### BRIEF DESCRIPTION OF THE DRAWINGS

FIG. 1 is a graph of the specific energy (in mJ/g) of treated seeds measured using the FT4 method described in Example 3, comparing wet and dry tableted talc. Lower bars indicate that less energy was required to complete the test, and indicate that the seeds exhibit increased flowability.

FIG. 2 is a graph of the specific energy (in mJ/g) of treated seeds measured using the FT4 method described in Example 3, comparing wet and dry tableted talc and talc dose.

FIG. 3 is a graph of the specific energy (in mJ/g) of treated seeds using the FT4 method described in Example 3, comparing talc grade and tableting pressure.

FIG. 4 is a graph of the specific energy (in mJ/g) of treated seeds, comparing pre-grinding of the talc and talc/mica blends.

FIG. 5 is a graph of the dust off (in grams dust per 100,000 seeds) of treated seeds using the method described in Example 4, comparing no talc, normal talc powder application, dry tableted talc, and wet tableted talc. Lower bars indicate lower dust generated.

FIG. 6 is a graph of the dust off (in grams dust per 100,000 seeds) of treated seeds using the method described in Example 4, comparing talc dosing on dry tableted talc and wet tableted talc.

### DETAILED DESCRIPTION

In general, the processes described herein are suitable for applying a seed treatment (e.g., a dry seed treatment) to exterior surfaces of seeds.

In some processes described herein, a single, solid one-piece body is provided. The single, solid one-piece body may comprise a seed treatment component, such as described below. The single, solid one-piece body is reduced. The seeds are brought into contact with the seed treatment component in the seed treater, which occurs simultaneously with the reduction of the single, solid one-piece body. The exemplary steps are not necessarily in the order listed above. Two or more of the steps may be performed simultaneously. Two or more of the steps may occur or be performed sequentially. Two or more of the steps may occur simultaneously, although it may not be necessary for the steps to begin and end at the same time in order to occur simultaneously. That is, to “occur simultaneously” two or more steps at least partially overlap in time, although initiation and/or completion of the steps may not be simultaneous.

Seed Treater

In some processes described herein, seeds are treated with the single, one-piece body in a seed treater. Suitable apparatuses and equipment (i.e., seed treaters) for treating seeds are known in the art, and include, without limitation, batch treaters, continuous treaters, drum and pan coaters, and fluid bed coaters. The seeds may be treated with the single, solid one-piece body in other types of machines, devices, and apparatuses.

Seeds and Plant Species

The seed treatment methods described herein can be used in connection with any species of plant and/or the seeds thereof. The methods are typically used in connection with seeds that are agronomically important. The seed may be a transgenic seed from which a transgenic plant can grow and incorporates a transgenic event that confers, for example, tolerance to a particular herbicide or combination of herbicides, increased disease resistance, enhanced tolerance to insects, drought, stress and/or enhanced yield. The seed may comprise a breeding trait, including for example, in one embodiment a disease tolerant breeding trait. In some instances, the seed includes at least one transgenic and breeding trait.

The process can be used for the treatment of any suitable seed type, including, but not limited to, row crops and vegetables. In some embodiments, one or more plants are selected from Amaranthaceae (e.g., chard, spinach, sugar beet, quinoa), Asteraceae (e.g., artichoke, asters, chamomile, chicory, chrysanthemums, dahlias, daisies, echinacea, goldenrod, guayule, lettuce, marigolds, safflower, sunflowers, zinnias), Brassicaceae (e.g., arugula, broccoli, bok choy, Brussels sprouts, cabbage, cauliflower, canola, collard greens, daikon, garden cress, horseradish, kale, mustard, radish, rapeseed, rutabaga, turnip, wasabi, watercress, Arabidopsis thaliana), Cucurbitaceae (e.g., cantaloupe, cucumber, honeydew, melon, pumpkin, squash (e.g., acorn squash, butternut squash, summer squash), watermelon, zucchini), Fabaceae (e.g., alfalfa, beans, carob, clover, guar, lentils, mesquite, peas, peanuts, soybeans, tamarind, tragacanth, vetch), Malvaceae (e.g., cacao, cotton, durian, hibiscus, kenaf, kola, okra), Poaceae (e.g., bamboo, barley, corn, fonio, lawn grass (e.g., Bahia grass, Bermudagrass, bluegrass, Buffalograss, Centipede grass, Fescue, or Zoysia), millet, oats, ornamental grasses, rice, rye, sorghum, sugar cane, triticale, wheat), Polygonaceae (e.g., buckwheat), Rosaceae (e.g., almonds, apples, apricots, blackberry, blueberry, cherries, peaches, plums, quinces, raspberries, roses, strawberries), Solanaceae (e.g., bell peppers, chili peppers, eggplant, petunia, potato, tobacco, tomato) and Vitaceae (e.g., grape).

Non-limiting examples of seeds that may be treated with compositions of the present disclosure include plants sold by Monsanto Company (St. Louis, Mo.) under the BOLLGARD DROUGHTGARD®, GENUITY®, RIB COMPLETE®, ROUNDUP READY®, ROUNDUP READY 2 YIELD®, ROUNDUP READY 2 EXTENDTM, SMARTSTAX®, VT DOUBLE PRO®, VT TRIPLE PRO®, YIELDGARD®, YIELDGARD VT ROOTWORM/RR2®, YIELDGARD VT TRIPLE® and/or XTENDFLEX™ tradenames.

Seed Treatment

In the processes described herein, a single, one-piece body comprising a seed treatment component is provided. As used herein, “providing” broadly means that the object (e.g., the single, one-piece body) is present in the seed treating process. The seed treatment component (i.e., at least one type of seed treatment component) of the single, one-piece body is suitable for providing some enhancement to the seeds, including but not limited to, enhanced growth, enhanced vigor, enhanced drought-resistance, enhanced handling, enhanced flowability, enhanced lubricity, enhanced yield enhanced germination, enhanced disease prevention, and enhanced insect prevention. The single, solid one-piece body has a selected mass and volume suitable for treating the seeds.

Seed Treatment Components

In some embodiments, the seed treatment component comprises a seed-finishing agent suitable for enhancing one or more physical properties of the exterior surfaces of the seeds. In some embodiments, the seed treatment component comprises a seed treatment active, such as biological agents and/or agrochemicals and/or other agents. The seed treatment component may comprise other seed treatment components for seed treating.

i. Seed-Finishing Agent

In the processes described herein, the seed treatment component may comprise a seed-finishing agent. In one or more examples, the seed-finishing agent may provide increased lubricity to the exterior surface of the seeds to aid in handling. In one or more examples, the seed-finishing agent may inhibit or reduce stickiness of the exterior surfaces of the seeds. In one or more examples, the seed-finishing agent may promote drying of the exterior surfaces of the seeds. In one or more examples, the seed-finishing agent may increase lubricity of the exterior surfaces of the seeds. In one or more examples, the seed-finishing agent may enhance uniformity of the exterior surfaces of the seeds. In one or more examples, the seed-finishing agent may reduce liquid load of the seed treater. It is understood that the seed component may include a single type of seed-finishing agent that provides one or more enhanced properties to the seeds. Alternatively or in addition, the seed component may comprise more than one type of seed-finishing agent that provides one or more enhanced properties to the seeds.

In some embodiments, the seed-finishing agent may comprise one or more minerals, including but not limited to talc, graphite, mica, and combinations thereof. Talc, graphite, and mica—whether alone or in combination with one another and/or other suitable agents—may be suitable for enhancing at least one of lubricity and/or flowability of the treated seeds, drying of the treated seeds, size uniformity of the seeds, and seed germination, among others. In some embodiments, the minerals comprise a mixture of talc and mica. For example, the talc to mica ratio may be about 60:40. In some embodiments, the minerals comprise a mixture of talc and graphite.

ii. Seed Treatment Active

In the processes described herein, the seed treatment may comprise a seed treatment active comprising one or more biological agents and/or agrochemicals and/or other agents. Non-limiting examples of useful biological agents include bacteria, fungi, beneficial nematodes, and viruses. Non-limiting examples of useful agrochemicals include pesticides, including fungicides, herbicides, insecticides, and nematicides. After being contacted by the seed treatment active, the seeds become “treated seeds,” as used herein.

The seed treatment active compositions and formulations in some embodiments may comprise one or more pesticidal agents. Pesticidal agents include chemical pesticides and biopesticides or biocontrol agents. Various types of chemical pesticides and biopesticides include acaricides, insecticides, nematicides, fungicides, gastropodicides, herbicides, virucides, bactericides, and combinations thereof. Biopesticides or biocontrol agents may include bacteria, fungi, beneficial nematodes, and viruses that exhibit pesticidal activity. Compositions of the present invention may comprise other agents for pest control, such as microbial extracts, plant growth activators, and/or plant defense agents.

Compositions in some embodiments may comprise one or more chemical acaricides, insecticides, and/or nematicides. Non-limiting examples of chemical acaricides, insecticides, and/or nematicides may include one or more carbamates, diamides, macrocyclic lactones, neonicotinoids, organophosphates, phenylpyrazoles, pyrethrins, spinosyns, synthetic pyrethroids, tetronic acids and/or tetramic acids. Non-limiting examples of chemical acaricides, insecticides, and nematicides that can be useful in compositions of the present disclosure include abamectin, acrinathrin, aldicarb, aldoxycarb, alpha-cypermethrin, betacyfluthrin, bifenthrin, cyhalothrin, cypermethrin, deltamethrin, csfenvalcrate, etofenprox, fenpropathrin, fenvalerate, flucythrinate, fosthiazate, lambda-cyhalothrin, gamma-cyhalothrin, permethrin, tau-fluvalinate, transfluthrin, zeta-cypermethrin, cyfluthrin, tefluthrin, eflusilanat, fubfenprox, pyrethrin, resmethrin, imidacloprid, acetamiprid, thiamethoxam, nitenpyram, thiacloprid, dinotefuran, clothianidin, imidaclothiz, chlorfluazuron, diflubenzuron, lufenuron, teflubenzuron, triflumuron, novaluron, flufenoxuron, hexaflumuron, bistrifluoron, noviflumuron, buprofezin, cyromazine, methoxyfenozide, tebufenozide, halofenozide, chromafenozide, endosulfan, fipronil, ethiprole, pyrafluprole, pyriprole, flubendiamide, chlorantraniliprole (e.g., Rynaxypyr), cyazypyr, emamectin, emamectin benzoate, abamectin, ivermectin, milbemectin, lepimectin, tebufenpyrad, fenpyroximate, pyridaben, fenazaquin, pyrimidifen, tolfenpyrad, dicofol, cyenopyrafen, cyflumetofen, acequinocyl, fluacrypyrin, bifenazate, diafenthiuron, etoxazole, clofentezine, spinosad, triarathen, tetradifon, propargite, hexythiazox, bromopropylate, chinomethionat, amitraz, pyrifluquinazon, pymetrozine, flonicamid, pyriproxyfen, diofenolan, chlorfenapyr, metaflumizone, indoxacarb, chlorpyrifos, spirodiclofen, spiromesifen, spirotetramat, pyridalyl, spinctoram, acephate, triazophos, profenofos, oxamyl, spinetoram, fenamiphos, fenamipclothiahos, 4-{[(6-chloropyrid-3-yl)methyl](2,2-difluoroethyl)amino}furan-2(5H)-one, 3,5-disubstituted-1,2,4-oxadiazole compounds, 3-phenyl-5-(thien-2-yl)-1,2,4-oxadiazole, cadusaphos, carbaryl, carbofuran, ethoprophos, thiodicarb, metamidophos, methiocarb, sulfoxaflor, cyantraniliprole and tioxazafen and combinations thereof. Additional non-limiting examples of chemical acaricides, insecticides, and/or nematicides may include one or more of abamectin, aldicarb, aldoxycarb, bifenthrin, carbofuran, chlorantraniliporle, chlothianidin, cyfluthrin, cyhalothrin, cypermethrin, cyantraniliprole, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, spinetoram, spinosad, spirodichlofen, spirotetramat, tefluthrin, thiacloprid, thiamethoxam and/or thiodicarb, and combinations thereof.

Additional non-limiting examples of acaricides, insecticides, and nematicides that may be included or used in compositions in some embodiments may be found in Steffey and Gray, Managing Insect Pests, ILLINOIS AGRONOMY HANDBOOK (2008); and Niblack, Nematodes, ILLINOIS AGRONOMY HANDBOOK (2008), the contents and disclosures of which are incorporated herein by reference. Non-limiting examples of commercial insecticides which may be suitable for the compositions disclosed herein include CRUISER (Syngenta, Wilmington, Del.), GAUCHO and PONCHO (Gustafson, Plano, Tex.). Active ingredients in these and other commercial insecticides may include thiamethoxam, clothianidin, and imidacloprid. Commercial acaricides, insecticides, and/or nematicides may be used in accordance with a manufacturer's recommended amounts or concentrations.

According to some embodiments, compositions in some embodiments may comprise one or more biopesticidal microorganisms, the presence and/or output of which is toxic to an acarid, insect and/or nematode. For example, compositions of the present invention may comprise one or more of Bacillus firmus I-1582, Bacillus mycoides AQ726, NRRL B-21664; Beauveria bassiana ATCC-74040, Beauveria bassiana ATCC-74250, Burkholderia sp. A396 sp. nov. rinojensis, NRRL B-50319, Chromobacterium subtsugae NRRL B-30655, Chromobacterium vaccinii NRRL B-50880, Flavobacterium H492, NRRL B-50584, Metarhizium anisopliae F52 (also known as Metarhizium anisopliae strain 52, Metarhizium anisopliae strain 7, Metarhizium anisopliae strain 43, and/or Metarhizium anisopliae BIO-1020, TAE-001; deposited as DSM 3884, DSM 3885, ATCC 90448, SD 170 and ARSEF 7711), Paecilomyces fumosoroseus FE991, and combinations thereof.

Compositions in some embodiments comprise one or more chemical fungicides. Non-limiting examples of chemical fungicides may include one or more aromatic hydrocarbons, benzthiadiazole, carboxylic acid amides, morpholines, phenylamides, phosphonates, thiazolidines, thiophene, quinone outside inhibitors and strobilurins, such as azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, 2-[2-(2,5-dimethyl-phenoxymethyl)-phenyl]-3-methoxy-acrylic acid methyl ester, and 2-(2-(3-(2,6-dichlorophenyl)-1-methyl-allylideneaminooxymethyl)-phenyl)-2-methoxyimino-N-methyl-acetamide, carboxamides, such as carboxanilides (e.g., benalaxyl, benalaxyl-M, benodanil, bixafen, boscalid, carboxin, fenfuram, fenhexamid, flutolanil, fluxapyroxad, furametpyr, isopyrazam, isotianil, kiralaxyl, mepronil, metalaxyl, metalaxyl-M (mefenoxam), ofurace, oxadixyl, oxycarboxin, penflufen, penthiopyrad, sedaxane, tecloftalam, thifluzamide, tiadinil, 2-amino-4-methyl-thiazole-5-carboxanilide, N-(4′-trifluoromethylthiobiphenyl-2-yl)-3-difluoromethyl-1-methyl-1H-pyrazole-4-carboxamide, N-(2-(1,3,3-trimethylbutyl)-phenyl)-1,3-dimethyl-5-fluoro-1H-pyrazole-4-carboxamide), carboxylic morpholides (e.g., dimethomorph, flumorph, pyrimorph), benzoic acid amides (e.g., flumetover, fluopicolide, fluopyram, zoxamide), carpropamid, dicyclomet, mandiproamid, oxytetracyclin, silthiofam, and N-(6-methoxy-pyridin-3-yl) cyclopropanecarboxylic acid amide, azoles, such as triazoles (e.g., azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole) and imidazoles (e.g., cyazofamid, imazalil, pefurazoate, prochloraz, triflumizol); heterocyclic compounds, such as pyridines (e.g., fluazinam, pyrifenox (cf.D1b), 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine), pyrimidines (e.g., bupirimate, cyprodinil, diflumetorim, fenarimol, ferimzone, mepanipyrim, nitrapyrin, nuarimol, pyrimethanil), piperazines (e.g., triforine), pirroles (e.g., fenpiclonil, fludioxonil), morpholines(e.g., aldimorph, dodemorph, dodemorph-acetate, fenpropimorph, tridemorph), piperidines (e.g., fenpropidin); dicarboximides (e.g., fluoroimid, iprodione, procymidone, vinclozolin), non-aromatic 5-membered heterocycles (e.g., famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester), acibenzolar-S-methyl, ametoctradin, amisulbrom, anilazin, blasticidin-S, captafol, captan, chinomethionat, dazomet, debacarb, diclomezine, difenzoquat, difenzoquat-methylsulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole and 5-chloro-7-(4-methylpiperidin-1-yl)-6-(2,4,6-trifluorophenyl)-[1,2,4]triazolo-[1,5-a]pyrimidine; benzimidazoles, such as carbendazim; and other active substances, such as guanidines (e.g., guanidine, dodine, dodine free base, guazatine, guazatine-acetate, iminoctadine), iminoctadine-triacetate and iminoctadine-tris(albesilate); antibiotics (e.g., kasugamycin, kasugamycin hydrochloride-hydrate, streptomycin, polyoxine and validamycin A), nitrophenyl derivates (e.g., binapacryl, dicloran, dinobuton, dinocap, nitrothal-isopropyl, tecnazen). organometal compounds (e.g., fentin salts, such as fentin-acetate, fentin chloride, fentin hydroxide); sulfur-containing heterocyclyl compounds (e.g., dithianon, isoprothiolane), organophosphorus compounds (e.g., edifenphos, fosetyl, fosetyl-aluminum, iprobenfos, phosphorus acid and its salts, pyrazophos, tolclofos-methyl), organochlorine compounds (e.g., chlorothalonil, dichlofluanid, dichlorophen, flusulfamide, hexachlorobenzene, pencycuron, pentachlorphenole and its salts, phthalide, quintozene, thiophanate-methyl, thiophanates, tolylfluanid, N-(4-chloro-2-nitro-phenyl)-N-ethyl-4-methyl-benzenesulfonamide) and inorganic active substances (e.g., Bordeaux mixture, copper acetate, copper hydroxide, copper oxychloride, basic copper sulfate, sulfur) and combinations thereof. In an aspect, compositions in some embodiments comprise acibenzolar-S-methyl, azoxystrobin, benalaxyl, bixafen, boscalid, carbendazim, cyproconazole, dimethomorph, epoxiconazole, fludioxonil, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thiabendazole, thifluzamide, thiophanate, tolclofos-methyl, trifloxystrobin and triticonazole, and combinations thereof.

For additional examples of fungicides that may be included in compositions in some embodiments, see, e.g., Bradley, Managing Diseases, ILLINOIS AGRONOMY HANDBOOK (2008), the content and disclosure of which are incorporated herein by reference.

Fungicides useful for compositions in some embodiments may exhibit activity against one or more fungal plant pathogens, including but not limited to Phytophthora, Rhizoctonia, Fusarium, Pythium, Phomopsis, Selerotinia or Phakopsora, and combinations thereof. Non-limiting examples of commercial fungicides which may be suitable for the compositions in some embodiments include PROTÉGÉ, RIVAL or ALLEGIANCE FL or LS (Gustafson, Plano, Tex.), WARDEN RTA (Agrilance, St. Paul, Minn.), APRON XL, APRON MAXX RTA or RFC, MAXIM 4FS or XL (Syngenta, Wilmington, Del.), CAPTAN (Arvesta, Guelph, Ontario) and PROTREAT (Nitragin Argentina, Buenos Ares, Argentina). Active ingredients in these and other commercial fungicides include, but are not limited to, fludioxonil, mefenoxam, azoxystrobin and metalaxyl. Commercial fungicides may be used in accordance with a manufacturer's recommended amounts or concentrations.

According to some embodiments, compositions in some embodiments may comprise one or more biopesticidal microorganisms, the presence and/or output of which is toxic to at least one fungus, bacteria, or both. For example, compositions of some embodiments may comprise one or more of Ampelomyces quisqualis AQ 10® (Intrachem Bio GmbH & Co. KG, Germany), Aspergillus flavus AFLA-GUARD® (Syngenta Crop Protection, Inc., CH), Aureobasidium pullulans BOTECTOR® (bio-ferm GmbH, Germany), Bacillus pumilus AQ717 (NRRL B-21662), Bacillus pumilus NRRL B-30087, Bacillus AQ175 (ATCC 55608), Bacillus AQ177 (ATCC 55609), Bacillus subtilis AQ713 (NRRL B-21661), Bacillus subtilis AQ743 (NRRL B-21665), Bacillus amyloliquefaciens FZB24, Bacillus amyloliquefaciens FZB42, Bacillus amyloliquefaciens NRRL B-50349, Bacillus amyloliquefaciens TJ1000 (also known as 1BE, isolate ATCC BAA-390), Bacillus subtilis ATCC 55078, Bacillus subtilis ATCC 55079, Bacillus thuringiensis AQ52 (NRRL B-21619), Candida oleophila I-182 (e.g., ASPIRE® from Ecogen Inc., USA), Candida saitoana BIOCURE® (in mixture with lysozyme; BASF, USA) and BIOCOAT® (ArystaLife Science, Ltd., Cary, N.C.), Clonostachys rosea f. catenulata (also referred to as Gliocladium catenulatum) J1446 (PRESTOP®, Verdera, Finland), Coniothyrium minitans CONTANS® (Prophyta, Germany), Cryphonectria parasitica (CNICM, France), Cryptococcus albidus YIELD PLUS® (Anchor Bio-Technologies, South Africa), Fusarium oxysporum BIOFOX® (from S.I.A.P.A., Italy) and FUSACLEAN® (Natural Plant Protection, France), Metschnikowia fructicola SHEMER® (Agrogreen, Israel), Microdochiurn dimerum ANTIBOT® (Agrauxine, France), Muscodor albus NRRL 30547, Muscodor roseus NRRL 30548, Phlebiopsis gigantea ROTSOP® (Verdera, Finland), Pseudozyma flocculosa SPORODEX® (Plant Products Co. Ltd., Canada), Pythium oligandrum DV74 (POLYVERSUM®, Remeslo SSRO, Biopreparaty, Czech Rep.), Reynoutria sachlinensis (e.g., REGALIA® from Marrone BioInnovations, USA), Streptomyces NRRL B-30145, Streptomyces M1064, Streptomyces galbus NRRL 30232, Streptomyces lydicus WYEC 108 (ATCC 55445), Streptomyces violaceusniger YCED 9 (ATCC 55660; DE-THATCH-9®, DECOMP-9® and THATCH CONTROL®, Idaho Research Foundation, USA), Streptomyces WYE 53 (ATCC 55750; DE-THATCH-9®, DECOMP-9® and THATCH CONTROL®, Idaho Research Foundation, USA), Talaromyces flavus V117b (PROTUS®, Prophyta, Germany), Trichoderma asperellum SKT-1 (ECO-HOPE®, Kumiai Chemical Industry Co., Ltd., Japan), Trichoderma atroviride LC52 (SENTINEL®, Agrimm Technologies Ltd, NZ), Trichoderma harzianum T-22 (PLANTSHIELD®, der Firma BioWorks Inc., USA), Trichoderma harzianum TH-35 (ROOT PRO®, from Mycontrol Ltd., Israel), Trichoderma harzianum T-39 (TRICHODEX®, Mycontrol Ltd., Israel; TRICHODERMA 2000®, Makhteshim Ltd., Israel), Trichoderma harzianum ICC012 and Trichoderma viride TRICHOPEL (Agrimm Technologies Ltd, NZ), Trichoderma harzianum ICC012 and Trichoderma viride ICC080 (REMEDIER® WP, Isagro Ricerca, Italy), Trichoderma polysporum and Trichoderma harzianum (BINAB®, BINAB Bio-Innovation AB, Sweden), Trichoderma stromaticum TRICOVAB® (C.E.P.L.A.C., Brazil), Trichoderma virens GL-21 (SOILGARD®, Certis LLC, USA), Trichoderma virens G1-3 (ATCC 57678), Trichoderma virens G1-21 (Thermo Trilogy Corporation, Wasco, Calif.), Trichoderma virens G1-3 and Bacillus amyloliquefaciens FZB24, Trichoderma virens G1-3 and Bacillus amyloliquefaciens NRRL B-50349, Trichoderma virens G1-3 and Bacillus amyloliquefaciens TJ1000, Trichoderma virens G1-21 and Bacillus amyloliquefaciens FZB24, Trichoderma vixens G1-21 and Bacillus amyloliquefaciens NRRL B-50349, Trichoderma virens G1-21 and Bacillus amyloliquefaciens TJ1000, Trichoderma viride TRIECO® (Ecosense Labs. (India) Pvt. Ltd., India, BIO-CURE® F from T. Stanes & Co. Ltd., Indien), Trichoderma viride TV1 (Agribiotec srl, Italy), Trichoderma viride ICC080, and/or Ulocladium oudemansii HRU3 (BOTRY-ZEN®, Botry-Zen Ltd, NZ), and combinations thereof.

Compositions in some embodiments may comprise one or more chemical herbicides. The herbicide may be a pre-emergent herbicide, a post-emergent herbicide, or a combination thereof. Non-limiting examples of chemical herbicides may comprise one or more acetyl CoA carboxylase (ACCase) inhibitors, acetolactate synthase (ALS) inhibitors, acetanilides, acetohydroxy acid synthase (AHAS) inhibitors, photosystem II inhibitors, photosystem I inhibitors, protoporphyrinogen oxidase (PPO or Protox) inhibitors, carotenoid biosynthesis inhibitors, enolpyruvylshikimate-3-phosphate (EPSP) synthase inhibitors, glutamine synthetase inhibitors, dihydropteroate synthetase inhibitors, mitosis inhibitors, 4-hydroxyphenyl-pyruvate-dioxygenase (4-HPPD) inhibitors, synthetic auxins, auxin herbicide salts, auxin transport inhibitors, nucleic acid inhibitors and/or one or more salts, esters, racemic mixtures and/or resolved isomers thereof. Non-limiting examples of chemical herbicides that can be useful in compositions of the present disclosure include 2,4-dichlorophenoxyacetic acid (2,4-D), 2,4,5-trichlorophenoxyacetic acid (2,4,5-T), ametryn, amicarbazone, aminocyclopyrachlor, acetochlor, acifluorfen, alachlor, atrazine, azafenidin, bentazon, benzofenap, bifenox, bromacil, bromoxynil, butachlor, butafenacil, butroxydim, carfentrazone-ethyl, chlorimuron, chlorotoluro, clethodim, clodinafop, clomazone, cyanazine, cycloxydim, cyhalofop, desmedipham, desmetryn, dicamba, diclofop, dimefuron, diuron, dithiopyr, fenoxaprop, fluazifop, fluazifop-P, fluometuron, flufenpyr-ethyl, flumiclorac, flumiclorac-pentyl, flumioxazin, fluoroglycofen, fluthiacet- methyl, fomesafen, fomesafen, glyphosate, glufosinate, halosulfuron, haloxyfop, hexazinone, imazamox, imazaquin, imazethapyr, ioxynil, isoproturon, isoxaflutole, lactofen, linuron, mecoprop, mecoprop-P, mesotrion, metamitron, metazochlor, methibenzuron, metolachlor (and S-metolachlor), metoxuron, metribuzin, monolinuron, oxadiargyl, oxadiazon, oxyfluorfen, phenmedipham, pretilachlor, profoxydim, prometon, prometry, propachlor, propanil, propaquizafop, propisochlor, pyraflufen-ethyl, pyrazon, pyrazolynate, pyrazoxyfen, pyridate, quizalofop, quizalofop-P (e.g., quizalofop-ethyl, quizalofop-P-ethyl, clodinafop-propargyl, cyhalofop-butyl, diclofop- methyl, fenoxaprop-P-ethyl, fluazifop-P-butyl, haloxyfop-methyl, haloxyfop-R-methyl), saflufenacil, sethoxydim, siduron, simazine, simetryn, sulcotrione, sulfentrazone, tebuthiuron, tembotrione, tepraloxydim, terbacil, terbumeton, terbuthylazine, thaxtomin (e.g., the thaxtomins described in U.S. Pat. No. 7,989,393), thenylchlor, tralkoxydim, triclopyr, trietazine, trifloxysulfuron, tropramezone, salts and esters thereof; racemic mixtures and resolved isomers thereof and combinations thereof. In an embodiment, compositions comprise acetochlor, clethodim, dicamba, flumioxazin, fomesafen, glyphosate, glufosinate, mesotrione, quizalofop, saflufenacil, sulcotrione, S-3100 and/or 2,4-D, and combinations thereof.

Additional examples of herbicides that may be included in compositions in some embodiments may be found in Hager, Weed Management, ILLINOIS AGRONOMY HANDBOOK (2008); and Loux et al., Weed Control Guide for Ohio, Indiana and Illinois (2015), the contents and disclosures of which are incorporated herein by reference. Commercial herbicides may be used in accordance with a manufacturer's recommended amounts or concentrations.

Compositions in some embodiments may comprise one or more virucides.

According to some embodiments, compositions in some embodiments may comprise one or more biopesticidal or herbicidal microorganisms, the presence and/or output of which is toxic to at least one insect, plant (weed), or phytopathogenic virus, as the case may be.

Additional examples of biopesticides that may be included or used in compositions in some embodiments may be found in BURGES, supra; HALL & MENN, BIOPESTICIDES: USE AND DELIVERY (Humana Press) (1998); McCoy et al., Entomogenous fungi, in CRC HANDBOOK OF NATURAL PESTICIDES. MICROBIAL PESTICIDES, PART A. ENTOMOGENOUS PROTOZOA AND FUNGI (C. M. Inoffo, ed.), Vol. 5:151-236 (1988); SAMSON et al., ATLAS OF ENTOMOPATHOGENIC FUNGI (Springer-Verlag, Berlin) (1988); and deFaria and Wraight, Mycoinsecticides and Mycoacaricides: A comprehensive list with worldwide coverage and international classification of formulation types, BIOL. CONTROL (2007), the contents and disclosures of which are incorporated herein by reference. In certain embodiments, a biocontrol microbe may comprise a bacterium of the genus Actinomycetes, Agrobacterium, Arthrobacter, Alcaligenes, Aureobacterium, Azobacter, Bacillus, Beijerinckia, Brevibacillus, Burkholderia, Chromobacterium, Clostridium, Clavibacter, Comamonas, Corynebacterium, Curtobacterium, Enterobacter, Flavobacterium, Gluconobacter, Hydrogenophaga, Klebsiella, Methylobacterium, Paenibacillus, Pasteuria, Photorhabdus, Phyllobacterium, Pseudomonas, Rhizobium, Serratia, Sphingobacterium, Stenotrophomonas, Variovorax, and Xenorhabdus, or any combination thereof. According to some embodiments, a biopesticidal microbe may include one or more of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens. According to some embodiments, a biopesticidal microbe may comprise a fungus of the genus Alternaria, Ampelomyces, Aspergillus, Aureobasidium, Beauveria, Colletotrichum, Coniothyrium, Gliocladium, Metarhizium, Muscodor, Paecilomyces, Trichoderma, Typhula, Ulocladium, and Verticillium. In another aspect a fungus is Beauveria bassiana, Coniothyrium minitans, Gliocladium virens, Muscodor albus, Paecilomyces lilacinus, or Trichoderma polysporum.

A composition in some embodiments may comprise one or more biocidal agents. A biocidal component may be included or used to prevent fungal and/or bacterial growth in the composition, particularly when the composition is placed in storage. Examples of biocidal agents include dichlorophen or benzyl alcohol hemiformal based compounds, benzoisothiazolinones and rhamnolipids. Non-limiting examples of commercially available biocidal agents include ACTICIDE (THOR), PROXEL (Arch Chemical), and ZONIX (Jeneil).

In addition to a microbial strain or isolate compositions and formulations in some embodiments may further comprise one or more agriculturally beneficial agents, such as biostimulants, nutrients, plant signal molecules, or biologically active agents.

According to some embodiments, compositions may comprise one or more beneficial biostimulants. Biostimulants may enhance metabolic or physiological processes such as respiration, photosynthesis, nucleic acid uptake, ion uptake, nutrient delivery, or a combination thereof. Non-limiting examples of biostimulants that may be included or used in compositions of the present invention may include seaweed extracts (e.g., Ascophyllum nodosum), bacterial extracts (e.g., extracts of one or more diazotrophs, phosphate-solubilizing microorganisms and/or biopesticides), fungal extracts, humic acids (e.g., potassium humate), fulvic acids, myo-inositol, and/or glycine, and any combinations thereof. According to some embodiments, the biostimulants may comprise one or more Azospirillum extracts (e.g., an extract of media comprising A. brasilense INTA Az-39), one or more Bradyrhizobium extracts (e.g., an extract of media comprising B. elkanii SEMIA 501, B. elkanii SEMIA 587, B. elkanii SEMIA 5019, B. japonicum NRRL B-50586 (also deposited as NRRL B-59565), B. japonicum NRRL B-50587 (also deposited as NRRL B-59566), B. japonicum NRRL B-50588 (also deposited as NRRL B-59567), B. japonicum NRRL B-50589 (also deposited as NRRL B-59568), B. japonicum NRRL B-50590 (also deposited as NRRL B-59569), B. japonicum NRRL B-50591 (also deposited as NRRL B-59570), B. japonicum NRRL B-50592 (also deposited as NRRL B-59571), B. japonicum NRRL B-50593 (also deposited as NRRL B-59572), B. japonicum NRRL B-50594 (also deposited as NRRL B-50493), B. japonicum NRRL B-50608, B. japonicum NRRL B-50609, B. japonicum NRRL B-50610, B. japonicum NRRL B-50611, B. japonicum NRRL B-50612, B. japonicum NRRL B-50726, B. japonicum NRRL B-50727, B. japonicum NRRL B-50728, B. japonicum NRRL B-50729, B. japonicum NRRL B-50730, B. japonicum SEMIA 566, B. japonicum SEMIA 5079, B. japonicum SEMIA 5080, B. japonicum USDA 6, B. japonicum USDA 110, B. japonicum USDA 122, B. japonicum USDA 123, B. japonicum USDA 127, B. japonicum USDA 129 and/or B. japonicum USDA 532C), one or more Rhizobium extracts (e.g., an extract of media comprising R. leguminosarum S012A-2), one or more Sinorhizobium extracts (e.g., an extract of media comprising S. fredii CCBAU114 and/or S. fredii USDA 205), one or more Penicillium extracts (e.g., an extract of media comprising P. bilaiae ATCC 18309, P. bilaiae ATCC 20851, P. bilaiae ATCC 22348, P. bilaiae NRRL 50162, P. bilaiae NRRL 50169, P. bilaiae NRRL 50776, P. bilaiae NRRL 50777, P. bilaiae NRRL 50778, P. bilaiae NRRL 50777, P. bilaiae NRRL 50778, P. bilaiae NRRL 50779, P. bilaiae NRRL 50780, P. bilaiae NRRL 50781, P. bilaiae NRRL 50782, P. bilaiae NRRL 50783, P. bilaiae NRRL 50784, P. bilaiae NRRL 50785, P. bilaiae NRRL 50786, P. bilaiae NRRL 50787, P. bilaiae NRRL 50788, P. bilaiae RS7B-SD1, P. brevicompactum AgRF18, P. canescens ATCC 10419, P. expansum ATCC 24692, P. expansum YT02, P. fellatanum ATCC 48694, P. gaestrivorus NRRL 50170, P. glabrum DAOM 239074, P. glabrum CBS 229.28, P. janthinellum ATCC 10455, P. lanosocoeruleum ATCC 48919, P. radicum ATCC 201836, P. radicum FRR 4717, P. radicum FRR 4719, P. radicum N93/47267 and/or P. raistrickii ATCC 10490), one or more Pseudomonas extracts (e.g., an extract of media comprising P. jessenii PS06), one or more acaricidal, insecticidal and/or nematicidal extracts (e.g., an extract of media comprising Bacillus firmus 1-1582, Bacillus mycoides AQ726, NRRL B-21664; Beauveria bassiana ATCC-74040, Beauveria bassiana ATCC-74250, Burkholderia sp. A396 sp. nov. rinojensis, NRRL B-50319, Chromobacterium subtsugae NRRL B-30655, Chromobacterium vaccinii NRRL B-50880, Flavobacterium H492, NRRL B-50584, Metarhizium anisopliae F52 (also known as Metarhizium anisopliae strain 52, Metarhizium anisopliae strain 7, Metarhizium anisopliae strain 43 and Metarhizium anisopliae BIO-1020, TAE-001; deposited as DSM 3884, DSM 3885, ATCC 90448, SD 170 and ARSEF 7711) and/or Paecilomyces fumosoroseus FE991), and/or one or more fungicidal extracts (e.g., an extract of media comprising Ampelomyces quisqualis AQ 10C) (Intrachem Bio GmbH & Co. KG, Germany), Aspergillus flavus AFLA-GUARD® (Syngenta Crop Protection, Inc., CH), Aureobasidium pullulans BOTECTOR® (bio-ferm GmbH, Germany), Bacillus pumilus AQ717 (NRRL B-21662), Bacillus pumilus NRRL B-30087, Bacillus AQ175 (ATCC 55608), Bacillus AQ177 (ATCC 55609), Bacillus subtilis AQ713 (NRRL B-21661), Bacillus subtilis AQ743 (NRRL B-21665), Bacillus amyloliquefaciens FZB24, Bacillus amyloliquefaciens NRRL B-50349, Bacillus amyloliquefaciens TJ1000 (also known as 1BE, isolate ATCC BAA-390), Bacillus thuringiensis AQ52 (NRRL B-21619), Candida oleophila 1-82 (e.g., ASPIRE® from Ecogen Inc., USA), Candida saitoana BIOCURE® (in mixture with lysozyme; BASF, USA) and BIOCOAT® (ArystaLife Science, Ltd., Cary, N.C.), Clonostachys rosea f. catenulata (also referred to as Gliocladium catenulatum) J1446 (PRESTOP®, Verdera, Finland), Coniothyrium minitans CONTANS® (Prophyta, Germany), Cryphonectria parasitica (CNICM, France), Cryptococcus albidus YIELD PLUS® (Anchor Bio-Technologies, South Africa), Fusarium oxysporum BIOFOX® (from S.I.A.P.A., Italy) and FUSACLEAN® (Natural Plant Protection, France), Metschnikowia fructicola SHEMER® (Agrogreen, Israel), Microdochiurn dimerum ANTIBOT® (Agrauxine, France), Muscodor albus NRRL 30547, Muscodor roseus NRRL 30548, Phlebiopsis gigantea ROTSOP® (Verdera, Finland), Pseudozyma flocculosa SPORODEX® (Plant Products Co. Ltd., Canada), Pythium oligandrum DV74 (POLYVERSUM®, Remeslo SSRO, Biopreparaty, Czech Rep.), Reynoutria sachlinensis (e.g., REGALIA® from Marrone BioInnovations, USA), Streptomyces NRRL B-30145, Streptomyces M1064, Streptomyces galbus NRRL 30232, Streptomyces lydicus WYEC 108 (ATCC 55445), Streptomyces violaceusniger YCED 9 (ATCC 55660; DE-THATCH-9®, DECOMP-9® and THATCH CONTROL®, Idaho Research Foundation, USA), Streptomyces WYE 53 (ATCC 55750; DE-THATCH-9®, DECOMP-9® and THATCH CONTROL®, Idaho Research Foundation, USA), Talaromyces flavus V117b (PROTUS®, Prophyta, Germany), Trichoderma asperellum SKT-1 (ECO-HOPE®, Kumiai Chemical Industry Co., Ltd., Japan), Trichoderma atroviride LC52 (SENTINEL®, Agrimm Technologies Ltd, NZ), Trichoderma harzianum T-22 (PLANTSHIELD®, der Firma BioWorks Inc., USA), Trichoderma harzianum TH-35 (ROOT PRO®, from Mycontrol Ltd., Israel), Trichoderma harzianum T-39 (TRICHODEX®, Mycontrol Ltd., Israel; TRICHODERMA 2000®, Makhteshim Ltd., Israel), Trichoderma harzianum ICC012 and Trichoderma viride TRICHOPEL (Agrimm Technologies Ltd, NZ), Trichoderma harzianum ICC012 and Trichoderma viride ICC080 (REMEDIER® WP, Isagro Ricerca, Italy), Trichoderma polysporum and Trichoderma harzianum (BINAB®, BINAB Bio-Innovation AB, Sweden), Trichoderma stromaticum TRICOVAB® (C.E.P.L.A.C., Brazil), Trichoderma vixens GL-21 (SOILGARD®, Certis LLC, USA), Trichoderma vixens G1-3, ATCC 57678, Trichoderma virens G1-21 (Thermo Trilogy Corporation, Wasco, Calif.), Trichoderma virens G1-3 and Bacillus amyloliquefaciens FZB2, Trichoderma virens G1-3 and Bacillus amyloliquefaciens NRRL B-50349, Trichoderma virens G1-3 and Bacillus amyloliquefaciens TJ1000, Trichoderma virens G1-21 and Bacillus amyloliquefaciens FZB24, Trichoderma virens G1-21 and Bacillus amyloliquefaciens NRRL B-50349, Trichoderma virens G1-21 and Bacillus amyloliquefaciens TJ1000, Trichoderma viride TRIECO® (Ecosense Labs. (India) Pvt. Ltd., Indien, BIO-CURE® F from T. Stanes & Co. Ltd., Indien), Trichoderma viride TV1 (Agribiotec srl, Italy), Trichoderma viride ICC080, and/or Ulocladium oudemansii HRU3 (BOTRY-ZEN®, Botry-Zen Ltd, NZ)), and combinations thereof.

Compositions in some embodiments may comprise one or more biologically active ingredients. Non-limiting examples of biologically active ingredients include plant growth regulators, plant signal molecules, growth enhancers, microbial stimulating molecules, biomolecules, soil amendments, nutrients, plant nutrient enhancers, etc., such as lipo-chitooligosaccharides (LCDs), chitooligosaccharides (COs), chitinous compounds, flavonoids, jasmonic acid or derivatives thereof (e.g., jasmonates), cytokinins, auxins, gibberellins, absiscic acid, ethylene, brassinosteroids, salicylates, macro- and micronutrients, linoleic acid or derivatives thereof, linolenic acid or derivatives thereof, karrikins, etc.) and beneficial microorganisms (e.g., Rhizobium spp., Bradyrhizobium spp., Sinorhizobium spp., Azorhizobium spp., Glomus spp., Gigaspora spp., Hymenoscyphous spp., Oidiodendron spp., Laccaria spp., Pisolithus spp., Rhizopogon spp., Scleroderma spp., Rhizoctonia spp., Acinetobacter spp., Arthrobacter spp, Arthrobotrys spp., Aspergillus spp., Azospirillum spp, Bacillus spp, Burkholderia spp., Candida spp., Chryseomonas spp., Enterobacter spp., Eupenicillium spp., Exiguobacterium spp., Klebsiella spp., Kluyvera spp., Microbacterium spp., Mucor spp., Paecilomyces spp., Paenibacillus spp., Penicillium spp., Pseudomonas spp., Serratia spp., Stenotrophomonas spp., Streptomyces spp., Streptosporangiurn spp., Swaminathania spp., Thiobacillus spp., Torulospora spp., Vibrio spp., Xanthobacter spp., Xanthomonas spp., etc.), and combinations thereof.

Compositions in some embodiments may comprise one or more lipo-chitooligosaccharides (LCDs), chitooligosaccharides (COs), and/or chitinous compounds. LCOs, sometimes referred to as symbiotic nodulation (Nod) signals (or Nod factors) or as Myc factors, consist of an oligosaccharide backbone of β-1,4-linked N-acetyl-D-glucosamine (“GlcNAc”) residues with an N-linked fatty acyl chain condensed at the non-reducing end. As understood in the art, LCOs differ in the number of GlcNAc residues in the backbone, in the length and degree of saturation of the fatty acyl chain and in the substitutions of reducing and non-reducing sugar residues. See, e.g., Denarie et al., Ann. Rev. Biochem. 65:503 (1996); Diaz et al., Mol. Plant-Microbe Interactions 13:268 (2000); Hungria et al., Soil Biol. Biochem. 29:819 (1997); Hamel et al., Planta 232:787 (2010); and Prome et al., Pure & Appl. Chem. 70(1):55 (1998), the contents and disclosures of which are incorporated herein by reference.

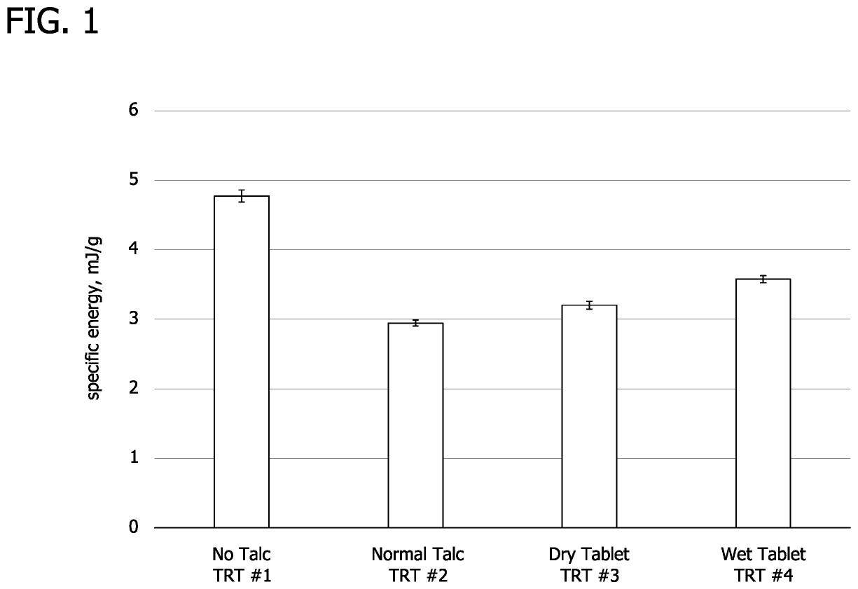

LCOs may be synthetic or obtained from any suitable source. See, e.g., WO 2005/063784, WO 2007/117500 and WO 2008/071674, the contents and disclosures of which are incorporated herein by reference. In some aspects, a synthetic LCO may have the basic structure of a naturally occurring LCO but contains one or more modifications or substitutions, such as those described in Spaink, Crit. Rev. Plant Sci. 54:257 (2000). LCOs and precursors for the construction of LCOs (e.g., COs, which may themselves be useful as a biologically active ingredient) can be synthesized by genetically engineered organisms. See, e.g., Samain et al., Carbohydrate Res. 302:35 (1997); Cottaz et al., Meth. Eng. 7(4):311 (2005); and Samain et al., J. Biotechnol. 72:33 (1999) (e.g., FIG. 1 therein, which shows structures of COs that can be made recombinantly in E. coli harboring different combinations of genes nodBCHL), the contents and disclosures of which are incorporated herein by reference.

LCOs (and derivatives thereof) may be included or utilized in compositions of the present invention in various forms of purity and can be used alone or in the form of a culture of LCO-producing bacteria or fungi. For example, OPTIMIZE® (commercially available from Monsanto Company (St. Louis, Mo.)) contains a culture of Bradyrhizobium japonicum that produces LCO. Methods to provide substantially pure LCOs include removing the microbial cells from a mixture of LCOs and the microbe, or continuing to isolate and purify the LCO molecules through LCO solvent phase separation followed by HPLC chromatography as described, for example, in U.S. Pat. No. 5,549,718. Purification can be enhanced by repeated HPLC and the purified LCO molecules can be freeze-dried for long-term storage. According to some embodiments, the LCO(s) included in compositions of the present disclosure is/are at least 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% pure. Compositions and methods in some embodiments may comprise analogues, derivatives, hydrates, isomers, salts and/or solvates of LCOs. LCOs may be incorporated into compositions of the present disclosure in any suitable amount(s)/concentration(s). For example, compositions of the present disclosure comprise about 1×10⁻²⁰ M to about 1×10⁻¹ M LCO(s). For example, compositions of the present disclosure can comprise about 1×10⁻²⁰ M, 1×10⁻¹⁹ M, 1×10⁻¹⁸ M, 1×10⁻¹⁷ M, 1×10⁻¹⁶ M, 1×10⁻¹⁵ M, 1×10⁻¹⁴ M, 1×10⁻¹³ M, 1×10⁻¹² M, 1×10⁻¹¹ M, 1×10⁻¹⁰ M, 1×10⁻⁹ M, 1×10⁻⁸ M, 1×10⁻⁷ M, 1×10⁻⁶ M, 1×10⁻⁵ M, 1×10⁻⁴ M, 1×10⁻³ M, 1×10⁻² M, 1×10⁻¹ M of one or more LCOs. In an aspect, the LCO concentration is 1×10⁻¹⁴ M to 1×10⁻⁵ M, 1×10⁻¹² M to 1×10⁻⁶ M, or 1×10⁻¹⁰ M to 1×10⁻⁷ M. In an aspect, the LCO concentration is 1×10⁻¹⁴ M to 1×10⁻⁵ M, 1×10⁻¹² M to 1×10⁻⁶ M, or 1×10⁻¹⁰ M to 1×10⁻⁷ M. The amount/concentration of LCO may be an amount effective to impart a positive trait or benefit to a plant, such as to enhance the disease resistance, growth and/or yield of the plant to which the composition is applied. According to some embodiments, the LCO amount/concentration is not effective to enhance the yield of the plant without beneficial contributions from one or more other constituents of the composition, such as CO and/or one or more pesticides.

Compositions in some embodiments may comprise any suitable COs, perhaps in combination with one or more LCOs. COs differ from LCOs in that they lack the pendant fatty acid chain that is characteristic of LCOs. COs, sometimes referred to as N-acetylchitooligosaccharides, are also composed of GlcNAc residues but have side chain decorations that make them different from chitin molecules [(C₈H₁₃NO₅)ₙ, CAS No. 1398-61-4] and chitosan molecules [(C₅H₁₁NO₄)ₙ, CAS No. 9012-76-4]. See, e.g., D'Haeze et al., Glycobiol. 12(6):79R (2002); Demont-Caulet et al., Plant Physiol. 120(1):83 (1999); Hanel et al., Planta 232:787 (2010); Muller et al., Plant Physiol. 124:733 (2000); Robina et al., Tetrahedron 58:521-530 (2002); Rouge et al., Docking of Chitin Oligomers and Nod Factors on Lectin Domains of the LysM-RLK Receptors in the Medicago-Rhizobium Symbiosis, in The Molecular Immunology of Complex Carbohydrates-3 (Springer Science, 2011); Van der Holst et al., Curr. Opin. Struc. Biol. 11:608 (2001); and Wan et al., Plant Cell 21:1053 (2009), the contents and disclosures of which are incorporated by reference. COs may be obtained from any suitable source. For example, the CO may be derived from an LCO. For example, in an aspect, compositions comprise one or more COs derived from an LCO obtained (i.e., isolated and/or purified) from a strain of Azorhizobium, Bradyrhizobium (e.g., B. japonicum), Mesorhizobium, Rhizobium (e.g., R. leguminosarum), Sinorhizobium (e.g., S. meliloti), or mycorhizzal fungi (e.g., Glomus intraradicus). Alternatively, the CO may be synthetic. Methods for the preparation of recombinant COs are known in the art. See, e.g., Cottaz et al., Meth. Eng. 7(4):311 (2005); Samain et al., Carbohydrate Res. 302:35 (1997); and Samain et al., J. Biotechnol. 72:33 (1999), the contents and disclosures of which are incorporated herein by reference.

COs (and derivatives thereof) may be included or utilized in compositions of the present invention in various forms of purity and can be used alone or in the form of a culture of CO-producing bacteria or fungi. According to some embodiments, the CO(s) included in compositions may be at least 0.1%, 0.5%, 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, 99.5% or more pure. It is to be understood that compositions and methods of the present disclosure can comprise hydrates, isomers, salts and/or solvates of COs. COs in some embodiments may be incorporated into compositions in any suitable amount(s)/concentration(s). For example, compositions in some embodiments may comprise about 1×10⁻²⁰ M to about 1×10⁻¹ M COs, such as about 1×10⁻²⁰ M, 1×10⁻¹⁹ M, 1×10⁻¹⁸ M, 1×10⁻¹⁷ M, 1×10⁻¹⁶ M, 1×10⁻¹⁵ M, 1×10⁻¹⁴ M, 1×10⁻¹³ M, 1×10⁻¹² M, 1×10⁻¹¹ M, 1×10⁻¹⁰ M, 1×10⁻⁹ M, 1×10⁻⁸ M, 1×10⁻⁷ M, 1×10⁻⁶ M, 1×10⁻⁵ M, 1×10⁻⁴ M, 1×10⁻³ M, 1×10⁻² M, or 1×10⁻¹ M of one or more COs. For example, the CO concentration may be 1×10⁻¹⁴ M to 1×10⁻⁵ M, 1×10⁻¹² M to 1×10⁻⁶ M, or 1×10⁻¹⁰ M to 1×10⁻⁷ M. The amount/concentration of CO may be an amount effective to impart or confer a positive trait or benefit to a plant, such as to enhance the soil microbial environment, nutrient uptake, or increase the growth and/or yield of the plant to which the composition is applied. Compositions in some embodiments may comprise one or more suitable chitinous compounds, such as, for example, chitin (IUPAC: N-[5-[[3-acetylamino-4,5-dihydroxy-6-(hydroxymethyl)oxan-2yl]methoxymethyl]-2-[[5-acetylamino-4,6-dihydroxy-2-(hydroxymethyl)oxan-3-yl]methoxymethyl]-4-hydroxy-6-(hydroxymethyl)oxan-3-ys]ethanamide), chitosan (IUPAC: 5-amino-6-[5-amino-6-[5-amino-4,6-dihydroxy-2(hydroxymethyl)oxan-3-yl]oxy-4-hydroxy-2-(hydroxymethyl)oxan-3-yl]oxy-2(hydroxymethyl)oxane-3,4-diol), and isomers, salts and solvates thereof.

Chitins and chitosans, which are major components of the cell walls of fungi and the exoskeletons of insects and crustaceans, are composed of GlcNAc residues. Chitins and chitosans may be obtained commercially or prepared from insects, crustacean shells, or fungal cell walls. Methods for the preparation of chitin and chitosan are known in the art. See, e.g., U.S. Pat. Nos. 4,536,207 (preparation from crustacean shells) and 5,965,545 (preparation from crab shells and hydrolysis of commercial chitosan); and Pochanavanich et al., Lett. Appl. Microbiol. 35:17 (2002) (preparation from fungal cell walls).

Deacetylated chitins and chitosans may be obtained that range from less than 35% to greater than 90% deacetylation and cover a broad spectrum of molecular weights, e.g., low molecular weight chitosan oligomers of less than 15 kD and chitin oligomers of 0.5 to 2 kD; “practical grade” chitosan with a molecular weight of about 15 kD; and high molecular weight chitosan of up to 70 kD. Chitin and chitosan compositions formulated for seed treatment are commercially available. Commercial products include, for example, ELEXA® (Plant Defense Boosters, Inc.) and BEYOND™ (Agrihouse, Inc.).

Compositions in some embodiments may comprise one or more suitable flavonoids, including, but not limited to, anthocyanidins, anthoxanthins, chalcones, coumarins, flavanones, flavanonols, flavans and isoflavonoids, as well as analogues, derivatives, hydrates, isomers, polymers, salts and solvates thereof. Flavonoids are phenolic compounds having the general structure of two aromatic rings connected by a three-carbon bridge. Classes of flavonoids are known in the art. See, e.g., Jain et al., J. Plant Biochem. & Biotechnol. 11:1 (2002); and Shaw et al., Environ. Microbiol. 11:1867 (2006), the contents and disclosures of which are incorporated herein by reference. Several flavonoid compounds are commercially available. Flavonoid compounds may be isolated from plants or seeds, e.g., as described in U.S. Pat. Nos. 5,702,752; 5,990,291; and 6,146,668. Flavonoid compounds may also be produced by genetically engineered organisms, such as yeast, See, e.g. Ralston et al., Plant Physiol. 137:1375 (2005).

According to embodiments, compositions may comprise one or more flavanones, such as one or more of butin, eriodictyol, hesperetin, hesperidin, homoeriodictyol, isosakuranetin, naringenin, naringin, pinocembrin, poncirin, sakuranetin, sakuranin, and/or sterubin, one or more flavanonols, such as dihydrokaempferol and/or taxifolin, one or more flavans, such as one or more flavan-3-ols (e.g., catechin (C), catechin 3-gallate (Cg), epicatechins (EC), epigallocatechin (EGC) epicatechin 3-gallate (ECg), epigallcatechin 3-gallate (EGCg), epiafzelechin, fisetinidol, gallocatechin (GC), gallcatechin 3-gallate (GCg), guibourtinidol, mesquitol, robinetinidol, theaflavin-3-gallate, theaflavin-3′-gallate, theflavin-3,3′-digallate, thearubigin), flavan-4-ols (e.g., apiforol and/or luteoforol) and/or flavan-3,4-diols (e.g., leucocyanidin, leucodelphinidin, leucofisetinidin, leucomalvidin, luecopelargonidin, leucopeonidin, leucorobinetinidin, melacacidin and/or teracacidin) and/or dimers, trimers, oligomers and/or polymers thereof (e.g., one or more proanthocyanidins), one or more isoflavonoids, such as one or more isoflavones or flavonoid derivatives (e.g, biochanin A, daidzein, formononetin, genistein and/or glycitein), isoflavanes (e.g., equol, ionchocarpane and/or laxifloorane), isoflavandiols, isoflavenes (e.g., glabrene, haginin D and/or 2-methoxyjudaicin), coumestans (e.g., coumestrol, plicadin and/or wedelolactone), pterocarpans, roetonoids, neoflavonoids (e.g, calophyllolide, coutareagenin, dalbergichromene, dalbergin, nivetin), and/or pterocarpans (e.g., bitucarpin A, bitucarpin B, erybraedin A, erybraedin B, erythrabyssin II, erthyrabissin-1, erycristagallin, glycinol, glyceollidins, glyceollins, glycyrrhizol, maackiain, medicarpin, morisianine, orientanol, phaseolin, pisatin, striatine, trifolirhizin), and combinations thereof. Flavonoids and their derivatives may be included in compositions in any suitable form, including, but not limited to, polymorphic and crystalline forms. Flavonoids may be included in compositions in any suitable amount(s) or concentration(s). The amount/concentration of a flavonoid(s) may be an amount effective to impart a benefit to a plant, which may be indirectly through activity on soil microorganisms or other means, such as to enhance plant nutrition and/or yield. According to some embodiments, a flavonoid amount/concentration may not be effective to enhance the nutrition or yield of the plant without the beneficial contributions from one or more other ingredients of the composition, such as LCO, CO, and/or one or more pesticides.

Compositions in some embodiments may comprise one or more suitable non-flavonoid nod-gene inducer(s), including, but not limited to, jasmonic acid ([1R-[1α,2β(Z)]]-3-oxo-2-(pentenyl)cyclopentaneacetic acid; JA), linoleic acid ((Z,Z)-9,12-Octadecadienoic acid) and/or linolenic acid ((Z,Z,Z)-9,12,15-octadecatrienoic acid), and analogues, derivatives, hydrates, isomers, polymers, salts and solvates thereof. Jasmonic acid and its methyl ester, methyl jasmonate (MeJA), collectively known as jasmonates, are octadecanoid-based compounds that occur naturally in some plants (e.g., wheat), fungi (e.g., Botryodiplodia theobromae, Gibbrella fujikuroi), yeast (e.g., Saccharomyces cerevisiae) and bacteria (e.g., Escherichia coli). Linoleic acid and linolenic acid may be produced in the course of the biosynthesis of jasmonic acid. Jasmonates, linoleic acid and linolenic acid (and their derivatives) are reported to be inducers of nod gene expression or LCO production by rhizobacteria. See, e.g., Mabood et al. PLANT PHYSIOL. BIOCHEM. 44(11):759 (2006); Mabood et al., AGR. J. 98(2):289 (2006); Mabood et al., FIELD CROPS RES. 95(2-3):412 (2006); and Mabood & Smith, Linoleic and linolenic acid induce the expression of nod genes in Bradyrhizobium japonicum USDA 3, PLANT BIOL. (2001).

Derivatives of jasmonic acid, linoleic acid, and linolenic acid that may be included or used in compositions in some embodiments include esters, amides, glycosides and salts thereof. Representative esters are compounds in which the carboxyl group of linoleic acid, linolenic acid, or jasmonic acid has been replaced with a —COR group, where R is an —OR¹ group, in which R¹ is: an alkyl group, such as a C₁-C₈ unbranched or branched alkyl group, e.g., a methyl, ethyl or propyl group; an alkenyl group, such as a C₂-C₈ unbranched or branched alkenyl group; an alkynyl group, such as a C₂-C₈ unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Representative amides are compounds in which the carboxyl group of linoleic acid, linolenic acid, or jasmonic acid has been replaced with a —COR group, where R is an NR²R³ group, in which R² and R³ are each independently: a hydrogen; an alkyl group, such as a C₁-C₈ unbranched or branched alkyl group, e.g., a methyl, ethyl or propyl group; an alkenyl group, such as a C₂-C₈ unbranched or branched alkenyl group; an alkynyl group, such as a C₂-C₈ unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Esters may be prepared by known methods, such as acid-catalyzed nucleophilic addition, wherein the carboxylic acid is reacted with an alcohol in the presence of a catalytic amount of a mineral acid. Amides may also be prepared by known methods, such as by reacting the carboxylic acid with the appropriate amine in the presence of a coupling agent, such as dicyclohexyl carbodiimide (DCC), under neutral conditions. Suitable salts of linoleic acid, linolenic acid and jasmonic acid include, for example, base addition salts. The bases that may be used as reagents to prepare metabolically acceptable base salts of these compounds include those derived from cations such as alkali metal cations (e.g., potassium and sodium) and alkaline earth metal cations (e.g., calcium and magnesium). These salts may be readily prepared by mixing a solution of linoleic acid, linolenic acid, or jasmonic acid with a solution of the base. The salts may be precipitated from solution and collected by filtration, or may be recovered by other means such as by evaporation of the solvent.

Non-flavonoid nod-gene inducers may be incorporated into compositions in any suitable amount(s)/concentration(s). For example, the amount/concentration of non-flavonoid nod-gene inducers may be an amount effective to impart or confer a positive trait or benefit to a plant, such as to enhance the disease resistance, growth and/or yield of the plant to which the composition is applied. According to some embodiments, the amount/concentration of non-flavonoid nod-gene inducers may not be effective to enhance the growth and/or yield of the plant without beneficial contributions from one or more other ingredients of the composition, such as a LCO, CO and/or one or more pesticides.

Compositions in some embodiments may comprise karrakins, including but not limited to 2H-furo[2,3-c]pyran-2-ones, as well as analogues, derivatives, hydrates, isomers, polymers, salts and solvates thereof. Examples of biologically acceptable salts of karrakins include acid addition salts formed with biologically acceptable acids, examples of which include hydrochloride, hydrobromide, sulphate or bisulphate, phosphate or hydrogen phosphate, acetate, benzoate, succinate, fumarate, maleate, lactate, citrate, tartrate, gluconate; methanesulphonate, benzenesulphonate and p-toluenesulphonic acid. Additional biologically acceptable metal salts may include alkali metal salts, with bases, examples of which include the sodium and potassium salts. Karrakins may be incorporated into compositions of the present invention in any suitable amount(s) or concentration(s). For example, the amount/concentration of a karrakin may be an amount or concentration effective to impart or confer a positive trait or benefit to a plant, such as to enhance the disease resistance, growth and/or yield of the plant to which the composition is applied. In an aspect, a karrakin amount/concentration may not be effective to enhance the disease resistance, growth and/or yield of the plant without beneficial contributions from one or more other ingredients of the composition, such as a LCO, CO and/or one or more pesticides.

iii. Other Seed Treatment Components

Generally, the seed treatment components described herein can also comprise one or more additional components. The additional component can be an additional ingredient, including for example, any adjuvants, excipients, nutrients, micronutrients, or other desirable components useful in seed treatment formulations. For example, in some embodiments, the seed treatment further comprises a surfactant.

Examples of anionic surfactants include alkyl sulfates, alcohol sulfates, alcohol ether sulfates, alpha olefin sulfonates, alkylaryl ether sulfates, arylsulfonates, alkylsulfonates, alkylaryl sulfonates, sulfosuccinates, mono- or diphosphate esters of polyalkoxylated alkyl alcohols or alkyl phenols, mono- or disulfosuccinate esters of alcohols or polyalkoxylated alkanols, alcohol ether carboxylates, phenol ether carboxylates. In one embodiment, the surfactant is an alkylaryl sulfonate.

Non-limiting examples of commercially available anionic surfactants include sodium dodecylsulfate (Na-DS, SDS), MORWET D-425 (a sodium salt of alkyl naphthalene sulfonate condensate, available from Akzo Nobel), MORWET D-500 (a sodium salt of alkyl naphthalene sulfonate condensate with a block copolymer, available from Akzo Nobel), sodium dodecylbenzene sulfonic acid (Na-DBSA) (available from Sigma Aldrich), diphenyloxide disulfonate, naphthalene formaldehyde condensate, DOWFAX (available from Dow), dihexylsulfosuccinate, and dioctylsulfosuccinate, alkyl naphthalene sulfonate condensates, and salts thereof.

Examples of non-ionic surfactants include sorbitan esters, ethoxylated sorbitan esters, alkoxylated alkylphenols, alkoxylated alcohols, block copolymer ethers, and lanolin derivatives. In accordance with one embodiment, the surfactant comprises an alkylether block copolymer.

Non-limiting examples of commercially available non-ionic surfactants include SPAN 20, SPAN 40, SPAN 80, SPAN 65, and SPAN 85 (available from Sigma Aldrich); TWEEN 20, TWEEN 40, TWEEN 60, TWEEN 80, and TWEEN 85 (available from Sigma Aldrich); IGEPAL CA-210, IGEPAL CA-520, IGEPAL CA-720, IGEPAL CO-210, IGEPAL CO-520, IGEPAL CO-630, IGEPAL CO-720, IGEPAL CO-890, and IGEPAL DM-970 (available from Sigma Aldrich); TRITON X-100 (available from Sigma Aldrich); BRIJ S10, BRIJ S20, BRIJ 30, BRIJ 52, BRIJ 56, BRIJ 58, BRIJ 72, BRIJ 76, BRIJ 78, BRIJ 92V, BRIJ 97, and BRIJ 98 (available from Sigma Aldrich); PLURONIC L-31, PLURONIC L-35, PLURONIC L-61, PLURONIC L-81, PLURONIC L-64, PLURONIC L-121, PLURONIC 10R5, PLURONIC 17R4, and PLURONIC 31R1 (available from Sigma Aldrich); Atlas G-5000 and Atlas G-5002L (available from Croda); ATLOX 4912 and ATLOX 4912-SF (available from Croda); SOLUPLUS (available from BASF); LANEXOL AWS (available from Croda); TRITON AG-98 (available from Rohm and Haas Co.); and Silwet L-77 (available from Momentive).

Non-limiting examples of cationic surfactants include mono alkyl quaternary amine, fatty acid amide surfactants, amidoamine, imidazoline, and polymeric cationic surfactants.

In some embodiments, the seed treatment active comprises a co-solvent in addition to water. Non-limiting examples of co-solvents that can be used include ethyl lactate, methyl soyate/ethyl lactate co-solvent blends (e.g., STEPOSOL, available from Stepan), isopropanol, acetone, 1,2-propanediol, n-alkylpyrrolidones (e.g., the AGSOLEX series, available from ISP), a petroleum based-oil (e.g., AROMATIC series and SOLVESSO series available from Exxon Mobil), isoparaffinic fluids (e.g. ISOPAR series, available from Exxon Mobil), cycloparaffinic fluids (e.g. NAPPAR 6, available from Exxon Mobil), mineral spirits (e.g. VARSOL series available from Exxon Mobil), and mineral oils (e.g., paraffin oil).

Examples of commercially available organic solvents include pentadecane, ISOPAR M, ISOPAR V, and ISOPAR L (available from Exxon Mobil).

Single, Solid One-Piece Body

In the processes described herein, the seed treatment process may comprise providing a single, solid one piece body (“SSOPB”). The SSOPB may be formed into a single, solid one-piece body. In some embodiments, the SSOPB comprises a seed treatment component, a seed treatment finishing agent, and/or an other seed treatment component. The SSOPB may be in the form of particles (e.g., powder or granules) compacted in a single, solid one-piece body. In some embodiments, the SSOPB may be in the form of particles (e.g., powder or granules) compacted in a single, solid one-piece body, with the seed treatment disposed in the single, solid one-piece body (e.g., interspersed with the non-treatment component particles). In some embodiments, the seed treatment is disposed in a matrix (e.g., matrix comprising gel, sugar, and/or a polymer) forming the solid one-piece body (e.g., interspersed with matrix).

In some embodiments, the SSOPB is molded into a suitable shape. For example, the SSOPB may be molded using compression molding, extrusion molding, rotational molding, and transfer molding. In some embodiments, the SSOPB may be self-assembled into a single, solid one-piece body. For example, the single, solid one-piece body may be formed through aggregation, coagulation, or flocculation of the seed treatment component. Where flocculation occurs, the solid flocs comprise the single, solid one-piece bodies of the seed treatment. In some embodiments, the solid flocs may be separated from the liquid component prior to contact with the seeds.

In some embodiments, the SSOPB comprises a seed-finishing agent comprising seed-finishing agent particles. The seed-finishing agent particles may have sizes from about 0.05 micrometer to about 100 micrometers, or from about 0.1 micrometer to about 50 micrometers, or from about 0.05 micrometers to about 60 micrometers. As an example, suitable talc particles may have mean particle sizes from about 0.05 micrometer to about 60 micrometers, or from about 0.1 micrometer to about 50 micrometers, or from about 0.05 micrometers to about 60 micrometers. As an example, suitable graphite particles may have mean particle sizes from about 0.05 micrometer to about 60 micrometers, or from about 0.1 micrometer to about 50 micrometers, or from about 0.05 micrometers to about 60 micrometers. As an example, suitable mica particles may have mean particle sizes from about 0.05 micrometer to about 60 micrometers, or from about 0.1 micrometer to about 50 micrometers, or from about 0.05 micrometers to about 60 micrometers.

In some embodiments, the seed-finishing agent particles, such as talc, graphite, and/or mica, may be formed into granules using wet granulation or dry granulation. For example, the granules may be generated by wet granulation of the dry talc, graphite, and/or mica powder, including use of a pan granulator, rotary bed granulator, and other techniques known to those in the art of wet granulation. The granules may be compacted into the single, solid one-piece body by compaction in a press or roll compactor. The single, solid one-piece body may be compacted using a pressure of 4 kgf/cm², 10 kgf/cm², 30 kgf/cm², 100 kgf/cm², 200 kgf/cm², and 300 kgf/cm².

In some embodiments, the SSOPB comprises a seed-finishing agent and a seed treatment active as described herein. In some embodiments, the SSOPB comprises a seed treatment active comprising one or more biological agents and/or agrochemicals and/or other agents is within the compacted seed-finishing agent particles forming the single, one-piece body.

In some embodiments, the SSOPB comprises a seed treatment active as described herein. In some embodiments, the SSOPB comprises a seed-finishing agent and a seed treatment active as described herein. In some embodiments, the SSOPB comprises a seed treatment active dispersed in a seed finishing agent matrix (e.g., matrix comprising gel, sugar, cellulose and/or a polymer) forming the solid one-piece body (e.g., interspersed with the seed finishing agent matrix).

In some embodiments, the SSOPB comprises a seed treatment active comprising seed treatment active particles. In some embodiments, the SSOPB comprises a seed treatment active compacted to form the single, one-piece body. In some embodiments, the SSOPB comprises an other seed treatment component compacted to form the single, one-piece body.

In some embodiments, the SSOPB comprises a seed-finishing agent and an other seed treatment component as described herein. In some embodiments, the SSOPB comprises an other seed treatment component compacted within the compacted seed-finishing agent particles forming the single, one-piece body.

In some embodiments, the SSOPB comprises a seed treatment active, a seed-finishing agent and an other seed treatment component as described herein. In some embodiments, the SSOPB comprises a seed treatment active, a seed-finishing agent and an other seed treatment component compacted forming the single, one-piece body. In other embodiments, the SSOPB comprises a seed treatment active, a seed-finishing agent and an other seed treatment component dispersed within a matrix.

The selected mass and volume of the SSOPB may be dependent on a number of factors, including but not limited to the types of seeds being treated, the number of seeds being treated, the type of seed treater, the composition of the seed treatment, and the desired extent of the treatment.

In some embodiments, the mass and volume of the SSOPB is suitable for enhancing, to a desired extent, one or more physical properties of the exterior surfaces of a selected amount of seeds. As an example, the SSOPB may have a mass from about 0.5 grams to about 400 grams. For example, the SSOPB may have a mass from about 0.5 grams to about 400 grams, or from about 10 grams to about 400 grams. As an example, the SSOPB may have a volume from about 10 cm³ to about 400 cm³. As an example, the SSOPB may have a density from about 0.00125 g/cm³ to about 40 g/cm³, for example, from about 0.9 g/cm³ to about 1.6 g/cm³.

In some embodiments, the shape and dimensions of the SSOPB is suitable for being added to a selected seed treater with the SSOPB being substantially whole and in a complete, non-separated form. The SSOPB may be any suitable shape and have any suitable dimensions. As described below, the shape and dimensions of the SSOPB may affect the ability of the single, solid one-piece body to be reduced, the reduced SSOPB being brought into contact with the seeds. The SSOPB may be in the shape of a sheet, a briquette, a disc, a pellet, or a tablet. In some embodiments, the SSOPB may not have any two dimensions exceeding 36 inches.

Additional Seed Treatment

In some embodiments, an additional seed treatment—apart from the SSOPB—may be applied to the seeds. The additional seed treatment may, for example, comprise any of the above seed-finishing agents, seed treatment actives, and other seed treatment components.

In some embodiments, the additional seed treatment comprises a liquid seed treatment, which may be a slurry. The liquid seed treatment may be in the form of an aqueous slurry comprising one or more dispersed solid phases and a continuous aqueous phase. In some instances, the liquid seed treatment composition further comprises a dispersed liquid organic phase. For example, the composition may be in the form of an aqueous suspension concentrate. In some embodiments, the separate seed treatment comprises a dry seed treatment. The dry seed treatment may be applied in addition to or as an alternative to the liquid seed treatment.

Application of the Additional Seed Treatment

Typically, when the process includes the application of an additional seed treatment, the seeds may be contacted with the additional seed treatment within the seed treatment apparatus (i.e., the seed treater).

The additional seed treatment can be applied to the seeds by a variety of means, for example by a spray nozzle or revolving disc, particularly when applying a liquid seed treatment. In some instances, as the seeds fall into the treatment apparatus, the seeds are treated (e.g., by misting or spraying with the liquid seed treatment active) and passed through the seed treater under continual movement, tumbling, and/or agitation. In some instances, the process comprises contacting the seed with the dry additional seed treatment during the same period in which the seed is contacted with the liquid seed treatment. In other instances, the seed is contacted with the dry seed treatment after the seed has been contacted with the liquid seed treatment.

When treating seeds on a large scale (for example a commercial scale), the additional seed treatment may be applied using a continuous process, a batch process, or a semi-batch process.

i. Continuous Processes

When the additional seed treatment is applied to the seeds using a continuous process, the seed treatment apparatus comprises a continuous seed treater. For example, in some instances, the additional seed treatment is applied using a continuous process and the seed treatment apparatus comprises a horizontal cylindrical drum. During the seed treatment process, the seeds may be mixed by tumbling due to the rotating motion of the drum.

In some instances, the seeds are contacted with the liquid seed treatment in a liquid application zone before they enter a horizontal cylindrical drum. The liquid application zone can be, for example, a mixer, including but not limited to a conical mixer. In other instances, the seeds are contacted with the additional seed treatment inside the horizontal cylindrical drum.

ii. Batch Processes

Alternatively, the seeds may be treated using a batch process. For example, a known weight of seeds can be introduced into the treatment equipment (such as a tumbler, a mixer, or a pan granulator). A known volume of the additional seed treatment can be introduced into the treatment equipment at a rate that allows the additional seed treatment to be applied evenly over the seeds. During the application, the seeds can be mixed, for example by spinning or tumbling.

When the additional seed treatment is applied to the seeds using a batch process, the first seed treatment apparatus may be, for example, a batch treater. For example, in some instances, the additional seed treatment is applied using a batch process and the seed treatment apparatus comprises a rotating bowl seed treater. In other instances, the additional seed treatment is applied using a batch process and the seed treatment apparatus comprises a rotating drum treater.

In a further alternative embodiment, the additional seed treatment may be applied using a semi-batch process that incorporates features from each of the batch process and continuous process embodiments set forth above.

Application of the SSOPB

The SSOPB is reduced (e.g., pulverized, ground, broken apart), and the seeds are brought into contact with the seed treatment. For example, the SSOPB may be reduced in the seed treater, and the seeds may be brought into contact with the seed treatment and/or SSOPB in the seed treater.

In some embodiments, reducing the SSOPB may overlap in time (i.e., occur simultaneously) with contacting the seeds with the seed treatment. In addition, reducing the SSOPB and contacting the seeds with the reduced SSOPB may occur together in the seed treater or another apparatus. For example, the SSOPB may be added in its complete form to the seeds in the seed treater. The SSOPB may be reduced upon addition to the seed treater and application of mechanical energy. As SSOPB is reduced in the seed treater, the SSOPB contacts the treated seeds. Mechanical energy for reducing the SSOPB may be the same mechanical energy for bringing the reduced SSOPB into contact with the seeds. The mechanical energy may be applied, for example, through rolling, agitation, or blending. The SSOPB may be reduced by any suitable means, including but not limited to pulverizing, grinding, crushing, compressing, cutting, shearing, and/or shaving.

In some embodiments, when reduction occurs, individual seed treatment component particles are formed. In these embodiments, the seed treatment component particles produced through application of mechanical energy in the seed treater may have a mean particle size of from about 1 micrometer to about 200 micrometers, or from about 0.05 micrometer to about 100 micrometer.

In other embodiments, where the SSOPB comprises a gel or wax, the gel or wax will be distributed among the seeds, thereby reducing the SSOPB without the formation of individual particles of the seed treatment component. In these embodiments, the seed treatment component may or may not be equally distributed among the seeds in the seed treater.

In some embodiments, reducing the seed treatment may be performed before contacting the seeds with the seed treatment component. For example, reduction of the SSOPB may take place outside the seed treater, or outside the location of the seeds in the seed treater, and then the reduced SSOPB may be added to the seeds in the seed treater.

Where the optional additional seed treatment is used, SSOPB of may be added to the seeds in the same seed treater that applies the additional seed treatment. In some embodiments, the reduction of the SSOPB may overlap in time (i.e., occur simultaneously) with the optional application of the additional seed treatment active. In some embodiments, the reduction of the SSOPB may occur after the optional additional seed treatment (e.g., a liquid seed treatment) is applied to the seeds, including when the treated seeds are in a semi-dry state.

In some embodiments, the SSOPB is added to the seeds when the seeds have a proper degree of surface wetness, such as after or during application of a liquid additional seed treatment, to promote good adhesion of the reduced seed treatment component to the surfaces of the seeds. This is particularly preferred where the seed treatment component of the reduced SSOPB is in the form of a powder, granules, or other dry composition. If the seeds are too wet when the dry composition is applied, the dry composition may agglomerate on the seeds and/or within the seed treater. On the other hand, if the seeds are allowed to become too dry before addition of the SSOPB, the seed treatment component may not adhere properly to the surfaces of the seeds, and the seeds may exhibit undesirable dust generation.

The surface wetness of the seeds can be routinely evaluated by those skilled in the art. For example, the surface wetness of the treated seeds can be tested using a glove test, wherein a sample of seeds taken from the treatment apparatus just before the application of the SSOPB step is held in a light-colored latex glove. If the glove becomes significantly colored with residue from the treated seeds, the process should be adjusted to provide the seeds with more spin time (in the case of a rotating bowl seed treater) or residence time (in the case of a horizontal drum seed treater) before they are contacted with the seed treatment.

Generally, there may be a significant range of surface wetness where the seed treatment application works satisfactorily, and an appropriate application point for the seed treatment can be determined by one skilled in the art using routine experimentation.

Additional Process Steps

Where an additional seed treatment is applied, the use of a single seed treatment apparatus for applying both the SSOPB and the additional seed treatment can provide several advantages, including a reduction in process complexity and/or a reduction in capital equipment costs. In some instances, however, the process comprises one or more additional steps following application of the additional seed treatment but before application of the SSOPB.

For example, in some instances, the process further comprises the use of a drying apparatus to dry the seeds. The use of a drying apparatus may be desirable, for example, in embodiments wherein a high application rate of the additional seed treatment is required.

Application Rates

Generally, the amount of the seed treatment component of the SSOPB that is applied to the seed can vary depending on the seed weight to be coated, surface area of the seed, the concentration of the agrochemical(s) and/or other active ingredients in the seed treatment active, the desired concentration on the finished seed, the plant species, and the environment in which the seed is intended to be sown, among other factors.

Similarly, the amount of the SSOPB applied to the seed depends upon the process parameters, crop type and content of the seed treatment composition, among other factors. In some embodiments, one dose of the SSOPB is applied to the seeds. In other embodiments, more than one dose of the SSOPB is applied to the seeds. Based on the quantity of seeds to be treated with the seed treatment, the mass of the SSOPB, and other factors, a skilled person would know how many SSOPB of the seed treatment to use.

Typically, upon reduction of the SSOPB through grinding, pulverization, or any other method of reduction outside of the treater, or application of mechanical energy inside of the seed treater, and after application to the seeds, the seeds will be coated with the reduced SSOPB (i.e., the seed treatment component). As an example, from about 0.1 grams to about 1 gram of the seed treatment component will be applied to 1 kilogram of seeds.

When the seed is a corn seed or a soybean seed, the seed treatment component is more typically applied in an amount from about 0.2 grams to about 0.75 grams per kilogram of seed.

Process Variables

Where the additional seed treatment is used, typically, the seed is contacted with the additional seed treatment for a duration of less than about 2 minutes. For example, in some instances, the seed may be contacted with the additional seed treatment for a duration of less than about 1 minute, less than about 45 seconds, less than about 30 seconds, or less than about 20 seconds. In this context, the term “contacting” refers to the period during which the additional seed treatment is introduced into the seed treatment apparatus.

For example, when the seed is a corn seed or a soybean seed, the seed is typically contacted with the additional seed treatment for an average duration of from about 45 seconds to about 90 seconds. When the seed is a cotton seed, it may be contacted with the additional seed treatment for an average duration of from about 60 seconds to about 120 seconds.

Typically, the wetted seed is contacted with the seed treatment for a duration of less than about 1 minute. For example, the wetted seed may be contacted with the seed treatment for a duration of less than about 45 seconds, less than about 30 seconds, or less than about 20 seconds. In this context, the term “contacting” refers to the period during which the seed treatment is introduced into the seed treatment apparatus.

For example, the wetted seed may be contacted with the seed treatment for an average duration of from about 20 seconds to about 60 seconds. During this time, the seed treatment may also be reducing into the reduced seed treatment.

Having described the invention in detail, it will be apparent that modifications and variations are possible without departing from the scope of the invention defined in the appended claims.

### EXAMPLES

The following non-limiting examples are provided to further illustrate the present invention.

### Example 1

### Preparation and Application of Seed Treatments of Compacted Talc Discs

Talc powder with a primary particle size distribution ranging from 0.1 to 50 micrometers was used. Fifty grams of the talc powder was weighed out into a circular die and pressed to approximately 14 kgf/cm² pressure and held for one minute with a hydraulic press. The compacted talc body was then removed from the die, cut into 7 gram pieces, and added to a seed treatment apparatus with one-unit of corn seeds (80,000 kernels). The seed treatment apparatus used was a Gustafson CBT 25. The seed treatment was added 25 seconds after the start of addition of the additional liquid seed treatment, and seed discharge was started at 40 seconds after the start of addition of the additional liquid seed treatment. The bowl was run at 70% speed (approximately 400 rpm).

Tablets were formed using wet granulation and dry granulation techniques. Dry tablets were prepared with a nominal mass of 50 g talc, while wet tablets were prepared by blending 50 g talc with 12.5 g DI water prior to compaction. Talc particles as described above, having a primary particle size distribution ranging from 0.1 to 50 micrometers, were compacted into tablets using pressures of approximately 4 kgf/cm², 14 kgf/cm², and 19 kgf/cm². Alternatively, talc particles with a primary particle size distribution ranging from 0.05 to 60 micrometers were compacted into tablets using a pressure of approximately 15 kfg/cm², 19 kgf/cm², 29 kgf/cm², and 39 kgf/cm². Both Arctic Mist and HCl 600 #1 grades of talc were used. Seeds were coated in the seed treatment apparatus in both half unit and full unit batches. Seeds were dosed with talc at 0.25 g talc per kg seed, 0.75 g talc per kg seed, 1.0 g talc per kg seed, and 1.9 g talc per kg seed.

### Example 2

### Preparation and Application of Seed Treatments of Mica Compacted Discs with Water

Thirty grams mica powder with a primary particle size distribution ranging from 0.04 to 50 micrometers was weighed out into a circular die, blended with 10 grams of deionized water, pressed to approximately 50 kgf/cm² pressure, and held for one minute with a hydraulic press. The wet compacted disc was then removed from the die. The moisture content was measured in an OHAUS MC 12 unit with a set point of 210° C. heating temperature and an endpoint condition of 0.1% moisture content lost per 120 seconds. Typically, a moisture content of ca. 25% moisture content is recovered. This is approximately the maximum moisture content which can be retained under 40 kgf/cm² compression.

The seed treatment apparatus used was a laboratory NIKLAS CBT. A mica compacted disc was added to each seed treatment apparatus at a dose of one gram per kilogram of seed. The compacted mica disc was added 15 seconds after the start of addition of the additional liquid seed treatment, and seed discharge was started at 30 seconds after the start of addition of the additional liquid seed treatment. The bowl was run at 40% drive speed.

### Example 3

### Preparation and Application of Seed Treatments of Compacted Mica Discs with Water Reduced to Coarse Particles by Mechanical Shear

Thirty grams mica powder with a primary particle size distribution ranging from 0.04 to 50 micrometers was weighed out into a circular die, blended with 10 grams of deionized water, and pressed to approximately 40 kgf/cm². Excess water was collected and removed outside the compression die. The wet compacted disc was then removed from the die.

The compacted disc of wetted mica was then milled to a coarse particle population consisting of particles with sizes ranging from approximately 0.1 mm to 10 mm. Optionally, the coarse particles were dried to remove excess moisture so as to improve the rate of mechanical disintegration during seed treatment. The minimum moisture content at which the solid compacted particle was retained was approximately 11% MC. Below this approximate moisture content, the solid particles were converted back into powder.

The seed treatment apparatus used was a laboratory NIKLAS CBT. Coarse compacted mica particles were added to the seed treatment apparatus at a dose of one gram mica per kilogram of seed. The coarse compacted mica particles were added 15 seconds after the start of addition of the additional liquid seed treatment, and seed discharge was started at 30 seconds after the start of addition of the additional liquid seed treatment. The bowl was run at 40% drive speed.

### Example 4

### Preparation and Application of Seed Treatment of Mica Granules with Water

One hundred grams mica powder with a primary particle size distribution ranging from 0.04 to 50 micrometers was weighed out into a container, blended with 60 grams of deionized water, and pressed with a paddle against a mesh containing 0.8 mm circular apertures through which the granules were pressed. Alternatively, one hundred grams of the mica powder was weighed out into a container and blended with an aqueous solution containing up to 4 weight percent of a PLURONIC surfactant. The surfactants used were PLURONIC L62 and PLURONIC P103.

The seed treatment apparatus used was a laboratory NIKLAS CBT. Mica granules were added to the seed treatment apparatus at a dose of one gram mica per kilogram of seed. The mica granules were added 15 seconds after the start of addition of the additional liquid seed treatment, and seed discharge was started at 30 seconds after the start of addition of the additional liquid seed treatment. The bowl was run at 40% drive speed.

### Example 5

### Comparison of Seed Treatments Based on Finishes with Different Types of Compacted Mica

Table 1 provides a detailed description of the seed treatments applied to the seeds and quantitative performance characteristics for seeds (DKC 64-34 AR2 1700 seeds per pound) treated with commercial fungicide and insecticide seed treatments. Appearance is not measured quantitatively for the purposes of the present disclosure. The composition volume was approximately 13.0 fluid ounces per one hundred pounds of seed. Seeds were treated at the batch=2 kilogram scale. Mica powder was dosed at 10 seconds dwell time (i.e., 10 seconds past the beginning of composition dosing), and total dwell time per batch was 25 seconds.

FT4 Measurement Protocol. Powder rheometry was used to quantify the decrease in seed-to-seed friction during bulk handling and transport. All experiments were performed on a FREEMAN TECHNOLOGY FT4 Powder Rheometer using a rotating 48.0 mm diameter impeller blade actuated through a pack of treated corn seeds. In all experiments, the impeller was actuated at 100 mm/s in all tests at a 5° helix angle. The seed pack depth was approximately 80 mm, 65 mm of which was traversed by the impeller blade during the experiment.

Dust-Off. Dust-off analysis was conducted using the HEUBACH Dust Meter.

TABLE 1

| | | Powder | Dust-off |
|---|---|---|---|
| Seed | | Rheology | (grams per |
| Treatment | Type | (mJ/gram seed) | 100,000 seeds) |
| A1 | No mica (negative control) | 3.22 | 0.023 |
| B1 | Compacted disc, 12% | 4.03 | 0.331 |
| | moisture content (1 g | | |
| | mica/kg seed) | | |
| C1 | Compacted disc, 25% | 3.81 | 0.250 |
| | moisture content (1 g | | |
| | mica/kg seed) | | |
| D1 | Granules, 12% moisture | 4.25 | 0.354 |
| | content (1 g mica/kg seed) | | |
| E1 | Granules, 18% moisture | 3.66 | 0.225 |
| | content (1 g mica/kg seed) | | |
| F1 | Mica powder (1 g mica/kg | 3.41 | 0.250 |
| | seed) | | |
| G1 | Coarse compacted particles, | 3.82 | 0.433 |
| | 12% moisture content (1 g | | |
| | mica/kg seed) | | |
| H1 | Coarse compacted particles, | 4.00 | 0.276 |
| | 18% moisture content (1 g | | |
| | mica/kg seed) | | |

### Example 6

### FT4 Flowability Tests

Seeds were treated with an additional liquid seed treatment and were dried at ambient temperature and humidity overnight. The seed treatments were prepared and applied using the procedure as set forth in Example 1 above.

The flowability of the seeds was measured using an FT4 powder rheometer with a 23.5 mm blade and a C2031 50 mm/160 mL borosilicate glass vessel No. 7762. The FT4 powder rheometer was used to measure the flow energy of each sample. Flow energy refers to the specific energy (in mJ/g) required to turn and move the probe through a column of the seeds, and is a measure of the flowability of the seeds. The less energy required to complete the test, the better (i.e., more easily) the seeds flowed.

Table 2 provides a detailed description of the talc seed treatments and the talc/mica seed treatments applied to the seeds.

TABLE 2

| Seed | |
|---|---|
| Treatment | Type |
| A2 | No talc (negative control) |
| B2 | Talc powder (0.75 g talc/kg seed) |
| C2 | Dry tableted talc (0.75 g talc/kg seed) |
| D2 | Wet tableted talc (0.75 g talc/kg seed) |
| E2 | Talc powder - low dose (0.25 g talc/kg seed) |
| F2 | Talc powder - medium dose (0.75 g talc/kg seed) |
| G2 | Talc powder - high dose (1.9 g talc/kg seed) |
| H2 | Dry tableted talc - low dose (0.25 g talc/kg seed) |
| I2 | Dry tableted talc - medium dose (0.75 g talc/kg seed) |
| J2 | Dry tableted talc - high dose (1.9 g talc/kg seed) |
| K2 | Wet tableted talc - low dose (0.25 g talc/kg seed) |
| L2 | Wet tableted talc - medium dose (0.75 g talc/kg seed) |
| M2 | Wet tableted talc - high dose (1.9 g talc/kg seed) |
| N2 | Arctic Mist talc (4.0 kgf/cm² pressure) |
| O2 | Arctic Mist talc (9.0 kgf/cm² pressure) |
| P2 | Arctic Mist talc (19.0 kgf/cm² pressure) |
| Q2 | HCl 600 #1 talc (4.0 kgf/cm² pressure) |
| R2 | HCl 600 #1 talc (9.0 kgf/cm² pressure) |
| S2 | Talc tablet (9 kgf/cm² pressure) |
| T2 | Talc tablet (22 kgf/cm² pressure + grinding) |
| U3 | Talc/mica (60:40, 15 kgf/cm² pressure) |
| V3 | Talc/mica (60:40, 22 kgf/cm² pressure) |

FIG. 1 is a graph illustrating the specific energy (in mJ/g) that was obtained for seed treatments as applied to corn seed, comparing dry tableted talc and wet tableted talc.

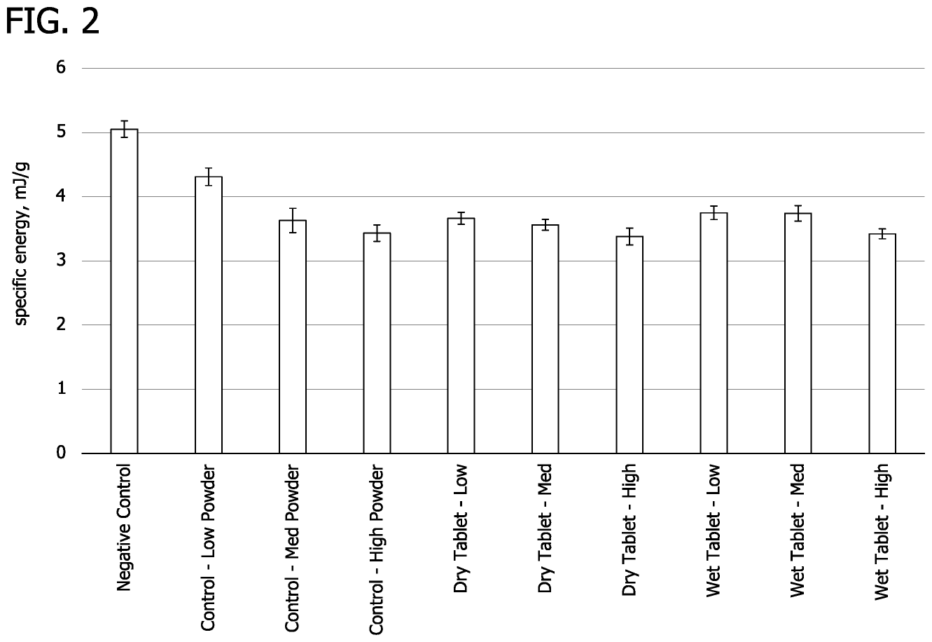

FIG. 2 is a graph illustrating the specific energy (in mJ/g) that was obtained for seed treatments as applied to corn seed, comparing dry tableted talc and wet tableted talc at different levels of talc dosing. This data indicates that low and high dosing levels yielded statistically different specific energies, but that dose types did not yield statistically different results.

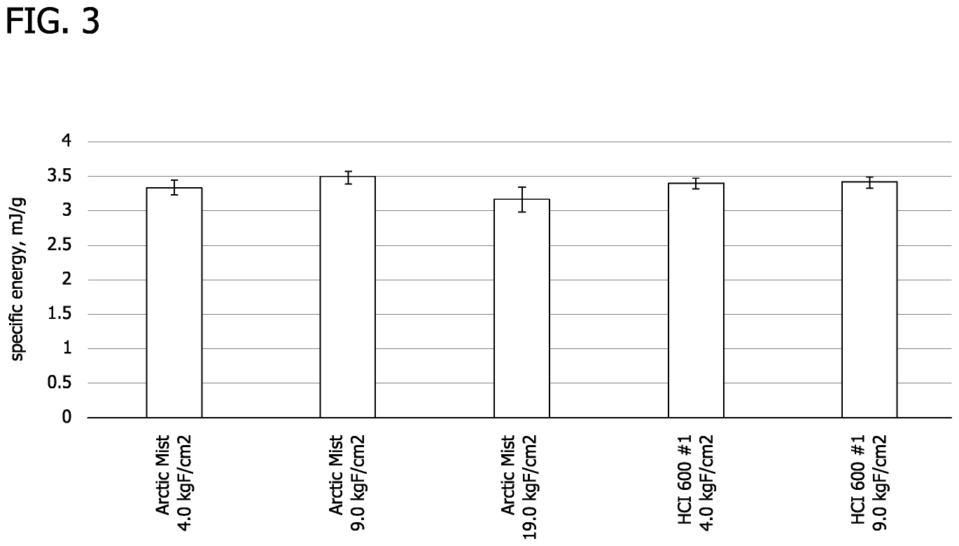

FIG. 3 is a graph illustrating the specific energy (in mJ/g) that was obtained for seed treatments as applied to corn seed, comparing compaction levels of Arctic Mist and HCl 600 #1 grades of talc. The results indicate that there is not a statistically significant variation in the response signal over the low to moderate compaction level regime. However, some compaction levels yield complete tablet breakup whereas others leave tablet pieces after reduction.

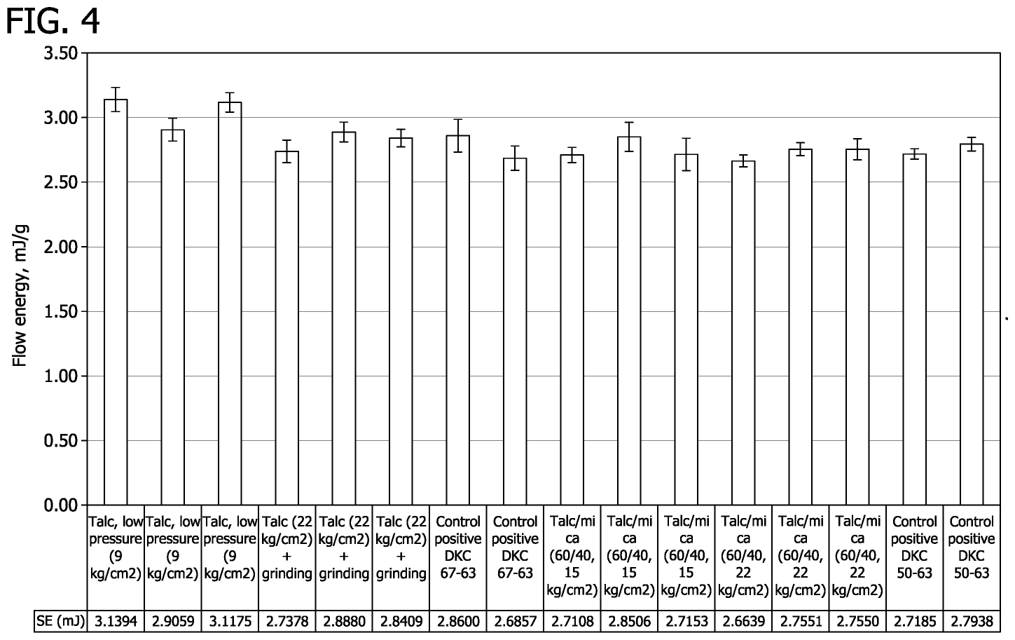

FIG. 4 is a graph illustrating the specific energy (in mJ/g) that was obtained for seed treatments as applied to corn seed, comparing talc with talc/mica blends.

As shown, seeds coated with the seed treatment exhibited a lower specific energy than the talc-free treatment, indicating a higher flowability. Dry granulation tablets and wet granulation tablets exhibited similar results and demonstrated lower specific energy than the talc-free treatment. Differences in talc dosing, tableting pressure, and talc to mica ratio did not substantially affect flowability.

### Example 7

### Dust-Off Tests

Seeds treated with an additional liquid seed treatment were prepared and were dried at ambient temperature and humidity overnight. The seed treatments were prepared and applied using the procedure as set forth in Example 1 above.

A HEUBACH Dust Meter was used to determine the amount of dust that can come off treated seed under stress and simulates mechanical stress after treatment (e.g., bagging, transport, and sowing).

Treated seed is mechanically stressed inside a rotating drum while a vacuum pump creates airflow through the rotating drum, glass cylinder, and attached filter unit. The airflow moves abraded particles out from the rotating drum through the glass cylinder and they are collected on the filter paper inside the filter unit. While floating dust particles settle on the filter, coarse non-floating particles are separated and collected in the glass cylinder. The amount of floating dust collected on the filter paper is determined gravimetrically.

The HEUBACH Dust Meter consists of a drive and control unit, a metal rotating drum, a glass cylinder, and a filter holding unit. A touch screen control panel allows setting of various parameters such as time of rotation (sec.), rotating speed (rpm) and airflow (ltr/min). The amount of abraded dust can be affected by the seed size. Therefore, when comparative studies are the done, the same seed size should be used whenever possible.

Before conducting the HEUBACH test, seed samples were conditioned for 48 hours at ambient lab temperature and humidity (or 20-25° C. and 30-50% relative humidity). The period of 48 hours is required to allow the seed coatings to completely dry especially if samples are taken immediately after the seed treatment process. Depending on circumstances, a shorter seed conditioning time may be acceptable, provided complete dryness of seed coating can be assured. Manufacturing seed samples for abrasion test should be taken at the bagging point.

Care should be taken when comparing samples to ensure that they have been treated and processed similarly. Samples obtained out of a lab treater and tested for dust will have different values than samples taken at the bagging station at a manufacturing site due to the downstream movement of seeds after the treatment.

The dust meter was turned on and allowed to warm up. The following parameters were selected: rotation speed: 30 rpm; air flow: 20 ltr/min; rotation time: 120 sec.; pre-selection: time. A sample of 100 (±1) grams of preconditioned treated seed was weighed and introduced into the rotating drum.

The filter unit was removed from the system. The filter unit was opened and a glass fiber filter paper (WHATMAN GF 92) was placed into the unit. The filter unit was then closed and weighed with an analytical balance to ±0.1 mg. The individual parts (rotating drum, glass cylinder, and filter unit) were assembled and the vacuum hose connected. After the rotating cycle had finished, the filter unit was disconnected and weighed. The difference of weight of the filter unit before and after the cycle was recorded and the weight of dust recalculated on the basis of grams dust/100 kg seed or grams dust/100,000 seeds taking into account the Thousand Grain Weight (TGW) of the seed sample tested.

The seeds in the drum after tumbling were weighed and the change in weight due to dust abrasion was calculated from the original weight. This gives an additional dust loss number compared to the value from the filter unit weight difference. The filter unit dust number is the HEUBACH dust value to be reported (i.e., the “floating” dust). Occasionally, the filter weighing will result in a negative number. In this case, the seed weight difference can be used to estimate the dust number.

A minimum of three replications is recommended for routine analysis. In case deviation between three replicates is too high, a fourth replicate should be performed. An untreated sample should also be included in any comparative study so that the dust baseline can be determined.

After each replication, all parts of the dust meter (drum, glass cylinder, and filter unit) were cleaned by washing with alcohol or acetone. Alternatively, a vacuum cleaner designed to handle toxic compounds can be used to clean the unit. Filters should be preserved for visual reference or forwarded to the analytical lab for active analysis.

Table 3 contains the dust-off data obtained during three runs of a single sample.

TABLE 3

| | | | | | Δ | Dust | |
|---|---|---|---|---|---|---|---|
| | | | Filter | Filter | Filter | (g)/100 | Avg Dust |
| | | Seed | Wt start | Wt end | Wt (g) | kg seed | (g)/100 |
| Sample ID | rep | Wt (g) | (g) | (g) | E − D | (F* 100000)/C | kg seed |
| 40001106437 | 1 | 100.1 | 0.1365 | 0.1371 | 0.0006 | 0.60 | 0.70 |
| 40001106437 | 2 | 100.07 | 0.1439 | 0.1448 | 0.0009 | 0.90 | |
| 40001106437 | 3 | 99.97 | 0.1458 | 0.1464 | 0.0006 | 0.60 | |

The precision (reproducibility) of the HEUBACH dust analysis method has been determined with both soybean and corn (Table 4). Results can be affected by how and where the sample is grabbed from the bulk seed. Dust values can also be affected by the size and shape of the seeds tested. The following data shows a representative set of data generated on a single corn sample (seed size AR; 1,592 seeds/lb.) from nine replicate runs through the HEUBACH dust meter.

TABLE 4

| Sample | Filter Pre | Filter Post | Δ filter | Dust (g)/ | | | |
|---|---|---|---|---|---|---|---|
| Wt (g) | Wt (g) | Wt (g) | wt (g) | 100 kg seed | Average | Std Dev | % RSD |
| 100.12 | 0.1411 | 0.1418 | 0.0007 | 0.6992 | | | |
| 100.14 | 0.1353 | 0.1357 | 0.0004 | 0.3994 | | | |
| 100.02 | 0.1396 | 0.1401 | 0.0005 | 0.4999 | | | |
| 100 | 0.1364 | 0.1368 | 0.0004 | 0.4000 | | | |
| 100 | 0.1396 | 0.1399 | 0.0003 | 0.3000 | | | |
| 100.06 | 0.1411 | 0.1415 | 0.0004 | 0.3998 | | | |
| 100.11 | 0.1333 | 0.1338 | 0.0005 | 0.4995 | | | |
| 100.08 | 0.1413 | 0.1418 | 0.0005 | 0.4996 | | | |
| 100.13 | 0.1325 | 0.133 | 0.0005 | 0.4994 | 0.4663 | 0.1116 | 23.9292 |

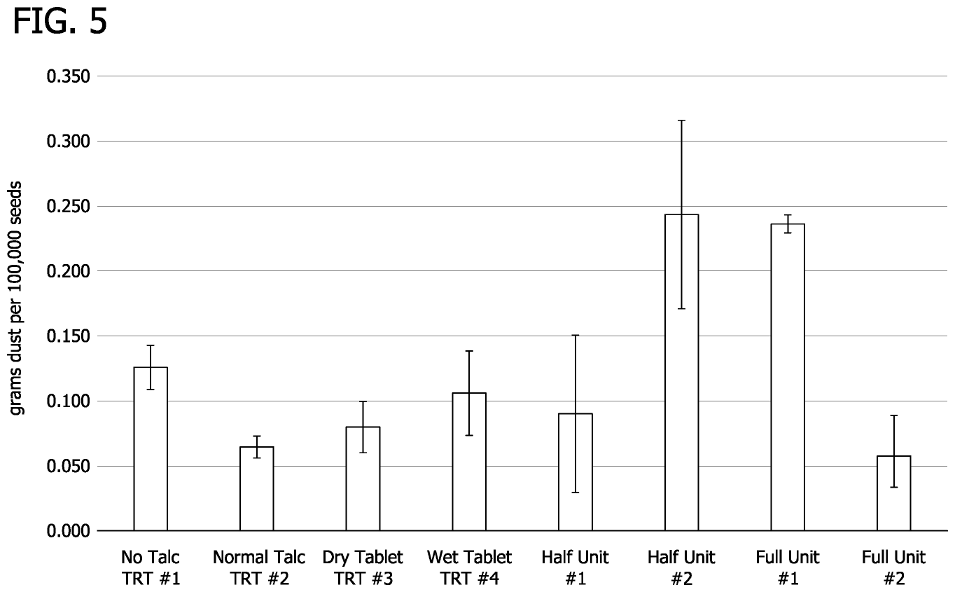

FIG. 5 is a graph illustrating the dust data (grams dust per 100,000 seeds) that were obtained for seed treatments as applied to a corn seed, comparing dry tableted talc and wet tableted talc.

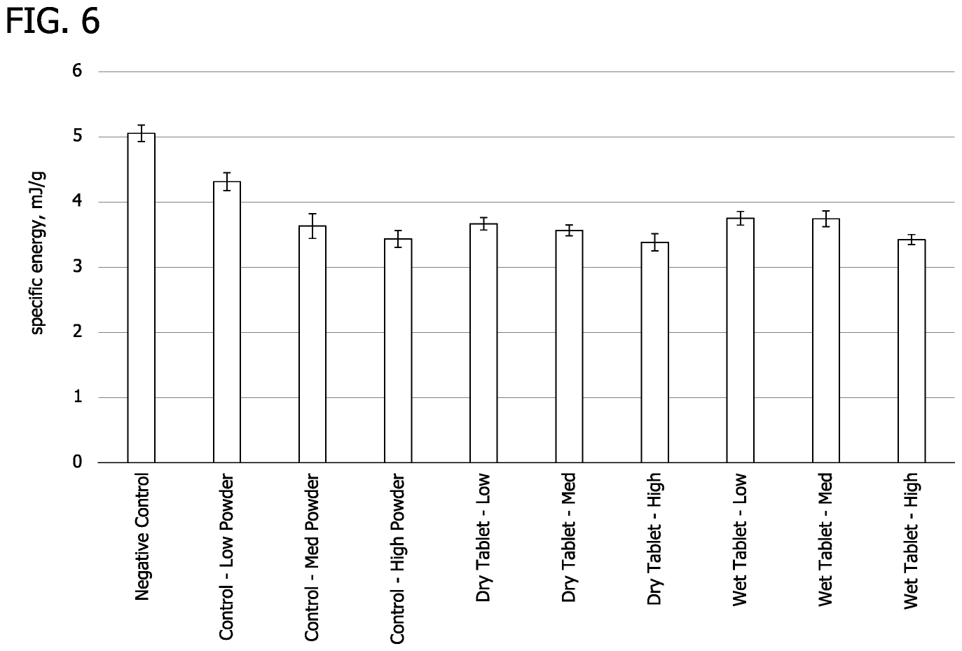

FIG. 6 is a graph illustrating the dust data (grams dust per 100,000 seeds) that were obtained for the seed treatments as applied to corn seed, comparing talc dosing. The data indicates a trend towards higher levels of dust-off at high talc dosing compared with dust levels at low talc dosing. However, dust-off measurements are known in the art to be imprecise. Given tight control over the relative humidity and temperature, it could be possible to reduce the relative standard deviation of the measurements. At the time the measurements were made, a 20% relative standard deviation was considered satisfactory. The data in FIG. 6 demonstrates that dust-off is statistically equivalent between the negative control, low and medium positive control, low dose of dry tableted talc, and low to medium dose of wet tableted talc.

### Example 8

### Evaluation of Moisture Loss and Moisture Content

A study is conducted to evaluation moisture loss and moisture content of a plurality of solid one-piece bodies contained in a vessel of specified water vapor transmission rate.

The solid one-piece bodies are comprised of rat least two components, one of these being moisture. The solid one-piece bodies are pre-blended so that the moisture content is substantially homogenous within the solid one-piece bodies,.

Additionally, the solid one-piece bodies are prepared with commercially available finishing agents, fungicides, insecticides, nematicides, and/or microorganisms. Test formulations are prepared by placing each component into a glass beaker followed by thorough mixing. The components of each test formulation are listed below in Tables 5A-5D.

TABLE 5A
Test Formulation A

| Addition Order | Component | Weight Fraction (%) |
|---|---|---|
| 1 | Finishing Agent A | 75.0 |
| 2 | Water | 25.0 |

TABLE 5B
Test Formulation B

| Addition Order | Component | Weight Fraction (%) |
|---|---|---|
| 1 | Finishing Agent A | 65.0 |
| 2 | Finishing Agent B | 10.0 |
| 3 | Water | 25.0 |

TABLE 5C
Test Formulation C

| Addition Order | Component | Weight Fraction (%) |
|---|---|---|
| 1 | Finishing Agent A | 70.0 |
| 2 | AEROSIL 300 | 7.0 |
| 3 | Water | 23.0 |

TABLE 5D
Test Formulation D

| Addition Order | Component | Weight Fraction (%) |
|---|---|---|
| 1 | Finishing Agent A | 75.0 |
| 2 | Microorganism A | 0.2 |
| 3 | Aqueous Dextrose | 24.8 |

Moisture content and tare weight are measured at time 0 and after 1 week, 2 weeks, 3 weeks, 4 weeks, 3 months, and 6 months of storage. Different samples are stored under standard conditions (20° C. and 50% relative humidity) and under accelerated aging conditions (50° C. oven). For each test formulation, 42 containers are prepared and marked with the sample ID, the storage condition, one of three container materials with varying degrees of water vapor transmission rate, and one of seven measurement types at noted in Table 6. Upon sealing the containment vessels after measurement at time 0, the vessels are stored at their respective environmental conditions until measurement, and are opened only once for moisture content measurement, and not at all for sample weight measurements.

TABLE 6
Sample Measurements

| Measurement Type | Measurement Time |
|---|---|
| Sample Weight | T0, Weeks 1, 2, 3, and 4, Months 3 and 6 |
| Moisture Content | T0, Week 1 |
| Moisture Content | T0, Week 2 |
| Moisture Content | T0, Week 3 |
| Moisture Content | T0, Week 4 |
| Moisture Content | T0, Month 3 |
| Moisture Content | T0, Month 6 |

### EMBODIMENTS

For further illustration, additional non-limiting embodiments of the present disclosure are set forth below.

For example, embodiment 1 is a method of preparing treated seeds, the method comprising: providing a single, solid one-piece body, wherein the single, solid one-piece body has a selected mass and volume; reducing the single, solid one-piece body; and contacting the seeds with the reduced single, solid one-piece body.

Embodiment 2 is the method of embodiment 1, wherein the single, solid one-piece body comprises a plurality of seed treatment component particles compacted into the single, solid one-piece body.

Embodiment 3 is the method of embodiment 2, wherein the plurality of seed treatment components particles are separated from one another after said reducing the single, solid one-piece body.

Embodiment 4 is the method of embodiment 3, wherein the separated seed treatment component particles have an average particle size from about 0.05 micrometer to about 100 micrometers.

Embodiment 5 is the method of any of embodiments 1 to 4, wherein said reducing the single, solid one-piece body is performed in a seed treater.

Embodiment 6 is the method of embodiment 5, wherein said reducing the single, solid one-piece body comprises applying mechanical energy to the single, solid one-piece body in the seed treater to break apart the single, solid one-piece body.

Embodiment 7 is the method of embodiment 6, wherein said applying mechanical energy comprises rolling, agitation, blending, or a combination thereof.

Embodiment 8 is the method of any one of embodiments 2 to7, wherein the seed treatment component particles comprises one or more types of minerals.

Embodiment 9 is the method of any one of embodiments 1 to 7, wherein the single, solid one-piece body comprises at least one of talc, graphite, mica, silica, starches, clays, celluloses, sugars, surfactants, and combinations thereof.

Embodiment 10 is the method of embodiment 8 or 9, wherein the single, solid one-piece body further comprises a binder, a finishing-agent promoter, or combinations thereof.

Embodiment 11 is the method of any one of embodiments 1 to 10, wherein the single, solid one-piece body is formed into particles through wet granulation or dry granulation.

Embodiment 12 is the method of any one of embodiments 1 to 11, wherein the single, solid one-piece body has the shape of a sheet, a briquette, a disc, a pellet, or a tablet.

Embodiment 13 is the method of any one of embodiments 1 to 12, wherein the seed treatment is formed into the single, solid one-piece body through compaction of particles, molding, aggregation, coagulation, or flocculation.

Embodiment 14 is the method of embodiment 13, wherein the compaction uses a pressure from about 4 kgf/cm² to about 300 kgf/cm².

Embodiment 15 is the method of any one of embodiments 1 to 14, wherein the mass of the single, solid one-piece body is from about 10 g to about 400 g.

Embodiment 16 is the method of any one of embodiments 1 to 15, wherein the volume of the single, solid one-piece body is from about 1 cm³ to about 100 cm³.

Embodiment 17 is the method of any one of embodiments 1 to 16, wherein the single, solid one-piece body has a density from about 0.9 g/cm³ to about 1.6 g/cm³.

Embodiment 18 is the method of any of embodiments 1 to 17, wherein the single, solid one-piece body comprises one or more biological agents, one or more agrochemicals, or combinations thereof.

Embodiment 19 is the method of any one of embodiments 1 to 18, further comprising applying a liquid slurry including one or more biological agents, one or more agrochemicals, or combinations thereof.

Embodiment 20 is the method of embodiment 19, wherein the single, solid one-piece body comprises one or more biological agents including one or more of bacterium, fungus, beneficial nematode, virus, and combinations thereof.

Embodiment 21 is the method of embodiment 18, wherein single, solid one-piece body comprises one or more agrochemicals including one or more of pesticide, fungicide, herbicide, insecticide, nematicide, and combinations thereof.

Embodiment 22 is the method of any of embodiments 1 to 21, wherein said reducing the single, solid one-piece body and said contacting the seeds with the reduced single, one piece body occur simultaneously.

Embodiment 23 is the method of any of embodiments 1 to 21, wherein the method comprises providing at least two single, solid one-piece bodies.

Embodiment 24 is a treated seed produced according to the method of any one of embodiments 1 to 22.

Modifications and variations of the disclosed embodiments are possible without departing from the scope of the invention defined in the appended claims.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A method of preparing treated seeds, the method comprising:
providing a single, solid one-piece body, wherein the single, solid one-piece body has a selected mass and volume;
reducing the single, solid one-piece body; and
contacting the seeds with the reduced single, solid one-piece body.

2. The method of claim 1, wherein the single, solid one-piece body comprises a plurality of seed treatment component particles compacted into the single, solid one-piece body.

3. The method of claim 2, wherein the plurality of seed treatment components particles are separated from one another after said reducing the single, solid one-piece body.

4. The method of claim 3, wherein the separated seed treatment component particles have an average particle size from about 0.05 micrometer to about 100 micrometers.

5. The method of any of claims 1 to 4, wherein said reducing the single, solid one-piece body is performed in a seed treater.

6. The method of claim 5, wherein said reducing the single, solid one-piece body comprises applying mechanical energy to the single, solid one-piece body in the seed treater to break apart the single, solid one-piece body.

7. The method of claim 6, wherein said applying mechanical energy comprises rolling, agitation, blending, or a combination thereof.

8. The method of any one of claims 2 to7, wherein the seed treatment component particles comprises one or more types of minerals.

9. The method of any one of claims 1 to 7, wherein the single, solid one-piece body comprises at least one of talc, graphite, mica, silica, starches, clays, celluloses, sugars, surfactants, and combinations thereof.

10. The method of claim 8 or 9, wherein the single, solid one-piece body further comprises a binder, a finishing-agent promoter, or combinations thereof.

11. The method of any one of claims 1 to 10, wherein the single, solid one-piece body component is formed into particles through wet granulation or dry granulation.

12. The method of any one of claims 1 to 11, wherein the single, solid one-piece body has the shape of a sheet, a briquette, a disc, a pellet, or a tablet.

13. The method of any one of claims 1 to 12, wherein the single, solid one-piece body is formed through compaction of particles, molding, aggregation, coagulation, or flocculation.

14. The method of claim 13, wherein the compaction uses a pressure from about 4 kgf/cm² to about 300 kgf/cm².

15. The method of any one of claims 1 to 14, wherein the mass of the single, solid one-piece body is from about 10 g to about 400 g.

16. The method of any one of claims 1 to 15, wherein the volume of the single, solid one-piece body is from about 1 cm³ to about 100 cm³.

17. The method of any one of claims 1 to 16, wherein the single, solid one-piece body has a density from about 0.9 g/cm³ to about 1.6 g/cm³.

18. The method of any of claims 1 to 17, wherein the single, solid one-piece body comprises one or more biological agents, one or more agrochemicals, or combinations thereof.

19. The method of any one of claims 1 to 18, further comprising applying a liquid slurry including one or more biological agents, one or more agrochemicals, or combinations thereof.

20. The method of claim 19, wherein the single, solid one-piece body comprises one or more biological agents including one or more of bacterium, fungus, beneficial nematode, virus, and combinations thereof.

21. The method of claim 18,wherein the single, solid one-piece body comprises one or more agrochemicals including one or more of pesticide, fungicide, herbicide, insecticide, nematicide, and combinations thereof.

22. The method of any of claims 1 to 21, wherein said reducing the single, solid one-piece body and said contacting the seeds with the reduced single, one piece body occur simultaneously.

23. The method of any of claims 1 to 21, wherein the method comprises providing at least two single, solid one-piece bodies.

24. A treated seed produced according to the method of any one of claims 1 to 23.
